SEQUENCE LISTING (153 sequences)

SEQ ID NO: 1 - DNA, 50 bp, Artificial Sequence
  fMet 1G (CAT)-5.F50 as ex.F primer
gtaatacgactcactataggcggggtggagcagcctggtagctcgtcggg

SEQ ID NO: 2 - DNA, 49 bp, Artificial Sequence
  Ala2 (GGC)-5.F49 as ex.F primer
gtaatacgactcactataggggctatagctcagctgggagagcgcttgc

SEQ ID NO: 3 - DNA, 50 bp, Artificial Sequence
  Arg2 (ACG->GCG)-5.F50 as ex.F primer
gtaatacgactcactatagcatccgtagctcagctggatagagtactcgg

SEQ ID NO: 4 - DNA, 49 bp, Artificial Sequence
  Asn 1G72C (GTT)-5.F49 as ex.F primer
gtaatacgactcactatagcctctgtagttcagtcggtagaacggcgga

SEQ ID NO: 5 - DNA, 50 bp, Artificial Sequence
  Asp (GTC)-5.F50 as ex.F primer
gtaatacgactcactataggagcggtagttcagtcggttagaatacctgc

SEQ ID NO: 6 - DNA, 48 bp, Artificial Sequence
  Cys (GCA)-5.F48 as ex.F primer
gtaatacgactcactataggcgcgttaacaaagcggttatgtagcgga

SEQ ID NO: 7 - DNA, 48 bp, Artificial Sequence
  Gln2 (CTG)-5l .F49 as ex.F primer
ggaacgcgcgactctaattggggtatcgccaagcggtaaggcaccgga

SEQ ID NO: 8 - DNA, 50 bp, Artificial Sequence
  Glu (TTC->CTC)-5.F50 as ex.F primer
gtaatacgactcactatagtccccttcgtctagaggcccaggacaccgcc

SEQ ID NO: 9 - DNA, 49 bp, Artificial Sequence
  Gly3 (GCC)-5.F49 as ex.F primer
gtaatacgactcactatagcgggaatagctcagttggtagagcacgacc

SEQ ID NO: 10 - DNA, 50 bp, Artificial Sequence
  His (GTG)-5.F50 as ex.F primer
gtaatacgactcactataggtggctatagctcagttggtagagccctgga

SEQ ID NO: 11 - DNA, 50 bp, Artificial Sequence
  Ile (GAT) 1G72C-5.F50 as ex.F primer
gtaatacgactcactatagggcttgtagctcaggtggttagagcgcaccc

SEQ ID NO: 12 - DNA, 50 bp, Artificial Sequence
  Leu2 (GAG)-5.F50 as ex.F primer
gtaatacgactcactatagccgaggtggtggaattggtagacacgctacc

SEQ ID NO: 13 - DNA, 49 bp, Artificial Sequence
  Lys (TTT->CTT)-5.F49 as ex.F primer
gtaatacgactcactatagggtcgttagctcagttggtagagcagttga

SEQ ID NO: 14 - DNA, 50 bp, Artificial Sequence
  Met (CAT)-5.F50 as ex.Fprimer
gtaatacgactcactataggctacgtagctcagttggttagagcacatca

SEQ ID NO: 15 - DNA, 49 bp, Artificial Sequence
  Phe 32C (GAA)-5.F49 as ex.F primer
gtaatacgactcactatagcccggatagctcagtcggtagagcagggga

SEQ ID NO: 16 - DNA, 50 bp, Artificial Sequence
  Pro2 (GGG)-5l.F50 as ex.F primer
ggaacgcgcgactctaatcggcacgtagcgcagcctggtagcgcaccgtc

SEQ ID NO: 17 - DNA, 50 bp, Artificial Sequence
  Ser5 (GGA)-5.F50 as ex.F primer
gtaatacgactcactataggtgaggtgtccgagtggctgaaggagcacgc

SEQ ID NO: 18 - DNA, 49 bp, Artificial Sequence
  Thr3 (GGT)-5.F49 as ex.F primer
gtaatacgactcactatagctgatatagctcagttggtagagcgcaccc

SEQ ID NO: 19 - DNA, 49 bp, Artificial Sequence
  Trp (CCA) -5l.F49 as ex.F primer
ggaacgcgcgactctaataggggcgtagttcaattggtagagcaccggt

SEQ ID NO: 20 - DNA, 50 bp, Artificial Sequence
  Tyr (GTA)-5.F50 as ex.F primer
gtaatacgactcactataggtggggttcccgagcggccaaagggagcaga

SEQ ID NO: 21 - DNA, 50 bp, Artificial Sequence
  Val2 (GAC)-5.F50 as ex.F primer
gtaatacgactcactatagcgtccgtagctcagttggttagagcaccacc

SEQ ID NO: 22 - DNA, 43 bp, Artificial Sequence
  fMet 1G (CAT)at.R43 as ex.R primer
gaaccgacgatcttcgggttatgagcccgacgagctaccaggc

SEQ ID NO: 23 - DNA, 43 bp, Artificial Sequence
  Ala2 (GGC)at.R43 as ex.R primer
gaaccgctgacctcttgcatgccatgcaagcgctctcccagct

SEQ ID NO: 24 - DNA, 43 bp, Artificial Sequence
  Arg2 (ACG->GCG)at.R43 as ex.R primer
gaacctccgaccgctcggttcgcagccgagtactctatccagc

SEQ ID NO: 25 - DNA, 43 bp, Artificial Sequence
  Asn 1G72C (GTT)at.R43 as ex.R primer
gaaccagtgacatacggattaacagtccgccgttctaccgact

SEQ ID NO: 26 - DNA, 43 bp, Artificial Sequence
  Asp (GTC)at.R43 as ex.R primer
gaacccgcgaccccctgcgtgacaggcaggtattctaaccgac

SEQ ID NO: 27 - DNA, 43 bp, Artificial Sequence
  Cys (GCA)at.R43 as ex.R primer
cgaaccggactagacggatttgcaatccgctacataaccgctt

SEQ ID NO: 28 - DNA, 43 bp, Artificial Sequence
  Gln2 (CTG)at.R43 as ex.R primer
gaacctcggaatgccggaatcagaatccggtgccttaccgctt

SEQ ID NO: 29 - DNA, 43 bp, Artificial Sequence
  Glu (TTC->CTC)at.R43 as ex.R primer
cgaacccctgttaccgccgtgagagggcggtgtcctgggcctc

SEQ ID NO: 30 - DNA, 43 bp, Artificial Sequence
  Gly3 (GCC)at.R43 as ex.R primer
gaactcgcgaccccgaccttggcaaggtcgtgctctaccaact

SEQ ID NO: 31 - DNA, 43 bp, Artificial Sequence
  His (GTG)at.R43 as ex.R primer
gaacccacgacaactggaatcacaatccagggctctaccaact

SEQ ID NO: 32 - DNA, 43 bp, Artificial Sequence
  Ile 1G72C (GAT)at.R43 as ex.R primer
gaaccaccgacctcacccttatcaggggtgcgctctaaccacc

SEQ ID NO: 33 - DNA, 43 bp, Artificial Sequence
  Leu2 (GAG)at.R43 as ex.R primer
gccctattgggcactaccacctcaaggtagcgtgtctaccaat

SEQ ID NO: 34 - DNA, 43 bp, Artificial Sequence
  Lys (TTT->CTT)at.R43 as ex.R primer
gaacctgcgaccaattgattaagagtcaactgctctaccaact

SEQ ID NO: 35 - DNA, 43 bp, Artificial Sequence
  Met (CAT)at.R43 as ex.R primer
gaacctgtgaccccatcattatgagtgatgtgctctaaccaac

SEQ ID NO: 36 - DNA, 43 bp, Artificial Sequence
  Phe 32C (GAA)at.R43 as ex.R primer
gaaccaaggacacggggattttcagtcccctgctctaccgact

SEQ ID NO: 37 - DNA, 43 bp, Artificial Sequence
  Pro2 (GGG)at.R43 as ex.R primer
gaacctccgacccccgacaccccatgacggtgcgctaccaggc

SEQ ID NO: 38 - DNA, 43 bp, Artificial Sequence
  Ser5 (GGA)at.R43 as ex.R primer
acgttgccgtatacacactttccaggcgtgctccttcagccac

SEQ ID NO: 39 - DNA, 43 bp, Artificial Sequence
  Thr3 (GGT)at.R43 as ex.R primer
gaactgccgacctcacccttaccaagggtgcgctctaccaact

SEQ ID NO: 40 - DNA, 43 bp, Artificial Sequence
  Trp (CCA)at.R43 as ex.R primer
gaactcccaacacccggttttggagaccggtgctctaccaatt

SEQ ID NO: 41 - DNA, 43 bp, Artificial Sequence
  Tyr (GTA)at.R43 as ex.R primer
gaagtctgtgacggcagatttacagtctgctccctttggccgc

SEQ ID NO: 42 - DNA, 43 bp, Artificial Sequence
  Val2 (GAC)at.R43 as ex.R primer
gaaccaccgacccccaccatgtcaaggtggtgctctaaccaac

SEQ ID NO: 43 - DNA, 43 bp, Artificial Sequence
  nLeu2S (GAG->GGA)at.R43 as ex.R primer
gccctattgggcactacctttcgagggtagcgtgtctaccaat

SEQ ID NO: 44 - DNA, 43 bp, Artificial Sequence
  nSer2L (CGA->GAG)at.R43 as ex.R primer
agttgcccctactccggtacctcaaaccggtccgttcagccgc

SEQ ID NO: 45 - DNA, 43 bp, Artificial Sequence
  nAla2L (GGC->GAG)at.R43 as ex.R primer
gaaccgctgacctcttgcacctcaagcaagcgctctcccagct

SEQ ID NO: 46 - DNA, 43 bp, Artificial Sequence
  nSer2A (CGA->GGC)at.R43 as ex.R primer
agttgcccctactccggtatgccataccggtccgttcagccgc

SEQ ID NO: 47 - DNA, 38 bp, Artificial Sequence
  fMet 1G (CAT)-3.R38 as PCR1.R primer
tggttgcgggggccggatttgaaccgacgatcttcggg

SEQ ID NO: 48 - DNA, 38 bp, Artificial Sequence
  Ala2 (GGC)-3.R38 as PCR1.R primer
tggtggagctaagcgggatcgaaccgctgacctcttgc

SEQ ID NO: 49 - DNA, 38 bp, Artificial Sequence
  Arg2 (ACG->GCG)-3.R38 as PCR1.R primer
tggtgcatccgggaggattcgaacctccgaccgctcgg

SEQ ID NO: 50 - DNA, 38 bp, Artificial Sequence
  Asn 1G72C (GTT)-3.R38 as PCR1.R primer
tggcgcctctgactggactcgaaccagtgacatacgga

SEQ ID NO: 51 - DNA, 38 bp, Artificial Sequence
  Asp (GTC)-3.R38 as PCR1.R primer
tggcggaacggacgggactcgaacccgcgaccccctgc

SEQ ID NO: 52 - DNA, 37 bp, Artificial Sequence
  Cys (GCA)-3.R37 as PCR1.R primer
tggaggcgcgttccggagtcgaaccggactagacgga

SEQ ID NO: 53 - DNA, 38 bp, Artificial Sequence
  Gln2 (CTG)-3.R38 as PCR1.R primer
tggctggggtacgaggattcgaacctcggaatgccgga

SEQ ID NO: 54 - DNA, 37 bp, Artificial Sequence
  Glu (TTC->CTC)-3.R37 as PCR1.R primer
tggcgtcccctaggggattcgaacccctgttaccgcc

SEQ ID NO: 55 - DNA, 38 bp, Artificial Sequence
  Gly3 (GCC)-3.R38 as PCR1.R primer
tggagcgggaaacgagactcgaactcgcgaccccgacc

SEQ ID NO: 56 - DNA, 38 bp, Artificial Sequence
  His (GTG)-3.R38 as PCR1.R primer
tggggtggctaatgggattcgaacccacgacaactgga

SEQ ID NO: 57 - DNA, 38 bp, Artificial Sequence
  Ile 1G72C(GAT)-3.R38 as PCR1.R primer
tggtgggcctgagtggacttgaaccaccgacctcaccc

SEQ ID NO: 58 - DNA, 48 bp, Artificial Sequence
  Leu2 (GAG)-3.R48 as PCR1.R primer
tggtaccgaggacgggacttgaacccgtaagccctattgggcactacc

SEQ ID NO: 59 - DNA, 38 bp, Artificial Sequence
  Lys (TTT->CTT)-3.R38 as PCR1.R primer
tggtgggtcgtgcaggattcgaacctgcgaccaattga

SEQ ID NO: 60 - DNA, 38 bp, Artificial Sequence
  Met (CAT)-3.R38 as PCR1.R primer
tggtggctacgacgggattcgaacctgtgaccccatca

SEQ ID NO: 61 - DNA, 38 bp, Artificial Sequence
  Phe 32C (GAA)-3.R38 as PCR1.R primer
tggtgcccggactcggaatcgaaccaaggacacgggga

SEQ ID NO: 62 - DNA, 38 bp, Artificial Sequence
  Pro2 (GGG)-3.R38 as PCR1.R primer
tggtcggcacgagaggatttgaacctccgacccccgac

SEQ ID NO: 63 - DNA, 51 bp, Artificial Sequence
  Ser2 (CGA)-3.R51 as PCR1.R primer
tggcggagagagggggatttgaacccccggtagagttgcccctactccggt

SEQ ID NO: 64 - DNA, 49 bp, Artificial Sequence
  Ser5 (GGA)-3.R49 as PCR1.R primer
tggcggtgagggggggattcgaacccccgatacgttgccgtatacacac

SEQ ID NO: 65 - DNA, 38 bp, Artificial Sequence
  Thr3 (GGT)-3.R38 as PCR1.R primer
tggtgctgataggcagattcgaactgccgacctcaccc

SEQ ID NO: 66 - DNA, 38 bp, Artificial Sequence
  Trp (CCA)-3.R38 as PCR1.R primer
tggcaggggcggagagactcgaactcccaacacccggt

SEQ ID NO: 67 - DNA, 46 bp, Artificial Sequence
  Tyr (GTA)-3.R46 as PCR1.R primer
tggtggtgggggaaggattcgaaccttcgaagtctgtgacggcaga

SEQ ID NO: 68 - DNA, 38 bp, Artificial Sequence
  Val2 (GAC)-3.R38 as PCR1.R primer
tggtgcgtccgagtggactcgaaccaccgacccccacc

SEQ ID NO: 69 - DNA, 20 bp, Artificial Sequence
  fMet 1G (CAT)-3.R20 as PCR2.R primer
tggttgcgggggccggattt

SEQ ID NO: 70 - DNA, 20 bp, Artificial Sequence
  Ala2 (GGC)-3.R20 as PCR2.R primer
tggtggagctaagcgggatc

SEQ ID NO: 71 - DNA, 20 bp, Artificial Sequence
  Arg2 (ACG->GCG)-3.R20 as PCR2.R primer
tggtgcatccgggaggattc

SEQ ID NO: 72 - DNA, 20 bp, Artificial Sequence
  Asn 1G72C (GTT)-3.R20 as PCR2.R primer
tggcgcctctgactggactc

SEQ ID NO: 73 - DNA, 20 bp, Artificial Sequence
  Asp (GTC)-3.R20 as PCR2.R primer
tggcggaacggacgggactc

SEQ ID NO: 74 - DNA, 20 bp, Artificial Sequence
  Cys (GCA)-3.R20 as PCR2.R primer
tggaggcgcgttccggagtc

SEQ ID NO: 75 - DNA, 20 bp, Artificial Sequence
  Gln2 (CTG)-3.R20 as PCR2.R primer
tggctggggtacgaggattc

SEQ ID NO: 76 - DNA, 20 bp, Artificial Sequence
  Glu (TTC->CTC)-3.R20 as PCR2.R primer
tggcgtcccctaggggattc

SEQ ID NO: 77 - DNA, 20 bp, Artificial Sequence
  Gly3 (GCC)-3.R20 as PCR2.R primer
tggagcgggaaacgagactc

SEQ ID NO: 78 - DNA, 20 bp, Artificial Sequence
  His (GTG)-3.R20 as PCR2.R primer
tggggtggctaatgggattc

SEQ ID NO: 79 - DNA, 20 bp, Artificial Sequence
  Ile 1G72C(GAT)-3.R20 as PCR2.R primer
tggtgggcctgagtggactt

SEQ ID NO: 80 - DNA, 20 bp, Artificial Sequence
  Leu2 (GAG)-3.R20 as PCR2.R primer
tggtaccgaggacgggactt

SEQ ID NO: 81 - DNA, 20 bp, Artificial Sequence
  Lys (TTT->CTT)-3.R20 as PCR2.R primer
tggtgggtcgtgcaggattc

SEQ ID NO: 82 - DNA, 20 bp, Artificial Sequence
  Met (CAT)-3.R20 as PCR2.R primer
tggtggctacgacgggattc

SEQ ID NO: 83 - DNA, 20 bp, Artificial Sequence
  Phe 32C (GAA)-3.R20 as PCR2.R primer
tggtgcccggactcggaatc

SEQ ID NO: 84 - DNA, 20 bp, Artificial Sequence
  Pro2 (GGG)-3.R20 as PCR2.R primer
tggtcggcacgagaggattt

SEQ ID NO: 85 - DNA, 20 bp, Artificial Sequence
  Ser2 (CGA)-3.R20 as PCR2.R primer
tggcggagagagggggattt

SEQ ID NO: 86 - DNA, 20 bp, Artificial Sequence
  Ser5 (GGA)-3.R20 as PCR2.R primer
tggcggtgagggggggattc

SEQ ID NO: 87 - DNA, 20 bp, Artificial Sequence
  Thr3 (GGT)-3.R20 as PCR2.R primer
tggtgctgataggcagattc

SEQ ID NO: 88 - DNA, 20 bp, Artificial Sequence
  Trp (CCA)-3.R20 as PCR2.R primer
tggcaggggcggagagactc

SEQ ID NO: 89 - DNA, 20 bp, Artificial Sequence
  Tyr (GTA)-3.R20 as PCR2.R primer
tggtggtgggggaaggattc

SEQ ID NO: 90 - DNA, 20 bp, Artificial Sequence
  Val2 (GAC)-3.R20 as PCR2.R primer
tggtgcgtccgagtggactc

SEQ ID NO: 91 - RNA, 77 nt, Artificial Sequence
  tRNA No. 0
ggcgggguggagcagccugguagcucgucgggcucauaacccgaagaucgucgguucaaauccggcccccgcaacca

SEQ ID NO: 92 - RNA, 76 nt, Artificial Sequence
  tRNA No. 1
ggggcuauagcucagcugggagagcgcuugcauggcaugcaagaggucagcgguucgaucccgcuuagcuccacca

SEQ ID NO: 93 - RNA, 77 nt, Artificial Sequence
  tRNA No. 2
gcauccguagcucagcuggauagaguacucggcugcgaaccgagcggucggagguucgaauccucccggaugcacca

SEQ ID NO: 94 - RNA, 76 nt, Artificial Sequence
  tRNA No. 3
gccucuguaguucagucgguagaacggcggacuguuaauccguaugucacugguucgaguccagucagaggcgcca

SEQ ID NO: 95 - RNA, 77 nt, Artificial Sequence
  tRNA No. 4
ggagcgguaguucagucgguuagaauaccugccugucacgcagggggucgcggguucgagucccguccguuccgcca

SEQ ID NO: 96 - RNA, 74 nt, Artificial Sequence
  tRNA No. 5
ggcgcguuaacaaagcgguuauguagcggauugcaaauccgucuaguccgguucgacuccggaacgcgccucca

SEQ ID NO: 97 - RNA, 75 nt, Artificial Sequence
  tRNA No. 6
ugggguaucgccaagcgguaaggcaccggauucugauuccggcauuccgagguucgaauccucguaccccagcca

SEQ ID NO: 98 - RNA, 76 nt, Artificial Sequence
  tRNA No. 7
guccccuucgucuagaggcccaggacaccgcccucucacggcgguaacagggguucgaauccccuaggggacgcca

SEQ ID NO: 99 - RNA, 76 nt, Artificial Sequence
  tRNA No. 8
gcgggaauagcucaguugguagagcacgaccuugccaaggucggggucgcgaguucgagucucguuucccgcucca

SEQ ID NO: 100 - RNA, 77 nt, Artificial Sequence
  tRNA No. 9
gguggcuauagcucaguugguagagcccuggauugugauuccaguugucguggguucgaaucccauuagccacccca

SEQ ID NO: 101 - RNA, 77 nt, Artificial Sequence
  tRNA No. 10
gggcuuguagcucaggugguuagagcgcaccccugauaagggugaggucggugguucaaguccacucaggcccacca

SEQ ID NO: 102 - RNA, 87 nt, Artificial Sequence
  tRNA No. 11
gccgaggugguggaauugguagacacgcuaccuugaggugguagugcccaauagggcuuacggguucaagucccguccucgguacca

SEQ ID NO: 103 - RNA, 76 nt, Artificial Sequence
  tRNA No. 12
gggucguuagcucaguugguagagcaguugacucuuaaucaauuggucgcagguucgaauccugcacgacccacca

SEQ ID NO: 104 - RNA, 77 nt, Artificial Sequence
  tRNA No. 13
ggcuacguagcucaguugguuagagcacaucacucauaaugauggggucacagguucgaaucccgucguagccacca

SEQ ID NO: 105 - RNA, 76 nt, Artificial Sequence
  tRNA No. 14
gcccggauagcucagucgguagagcaggggacugaaaauccccguguccuugguucgauuccgaguccgggcacca

SEQ ID NO: 106 - RNA, 77 nt, Artificial Sequence
  tRNA No. 15
cggcacguagcgcagccugguagcgcaccgucauggggugucgggggucggagguucaaauccucucgugccgacca

SEQ ID NO: 107 - RNA, 88 nt, Artificial Sequence
  tRNA No. 16
ggugagguguccgaguggcugaaggagcacgccuggaaaguguguauacggcaacguaucggggguucgaaucccccccucaccgcca

SEQ ID NO: 108 - RNA, 76 nt, Artificial Sequence
  tRNA No. 17
gcugauauagcucaguugguagagcgcacccuugguaagggugaggucggcaguucgaaucugccuaucagcacca

SEQ ID NO: 109 - RNA, 76 nt, Artificial Sequence
  tRNA No. 18
aggggcguaguucaauugguagagcaccggucuccaaaaccggguguugggaguucgagucucuccgccccugcca

SEQ ID NO: 110 - RNA, 85 nt, Artificial Sequence
  tRNA No. 19
ggugggguucccgagcggccaaagggagcagacuguaaaucugccgucacagacuucgaagguucgaauccuucccccaccacca

SEQ ID NO: 111 - RNA, 77 nt, Artificial Sequence
  tRNA No. 20
gcguccguagcucaguugguuagagcaccaccuugacauggugggggucggugguucgaguccacucggacgcacca

SEQ ID NO: 112 - RNA, 87 nt, Artificial Sequence
  tRNA No. 21
gccgaggugguggaauugguagacacgcuacccuggaaagguagugcccaauagggcuuacggguucaagucccguccucgguacca

SEQ ID NO: 113 - RNA, 90 nt, Artificial Sequence
  tRNA No. 22
ggagagaugccggagcggcugaacggaccgguuugagguaccggaguaggggcaacucuaccggggguucaaaucccccucucuccgcca

SEQ ID NO: 114 - RNA, 76 nt, Artificial Sequence
  tRNA No. 23
ggggcuauagcucagcugggagagcgcuugcuugaggugcaagaggucagcgguucgaucccgcuuagcuccacca

SEQ ID NO: 115 - RNA, 90 nt, Artificial Sequence
  tRNA No. 24
ggagagaugccggagcggcugaacggaccgguauggcauaccggaguaggggcaacucuaccggggguucaaaucccccucucuccgcca

SEQ ID NO: 116 - DNA, 20 bp, Artificial Sequence
  pGEM-Tseq.R20 as Primer
ggaaacagctatgaccatga

SEQ ID NO: 117 - DNA, 22 bp, Artificial Sequence
  M1RNA.R22 as Primer
aggtgaaactgaccgataagcc

SEQ ID NO: 118 - DNA, 36 bp, Artificial Sequence
  T7pro-leader.F36 as Primer
gtaatacgactcactataggaacgcgcgactctaat

SEQ ID NO: 119 - DNA, 22 bp, Artificial Sequence
  T7ex5.F22 as Primer
ggcgtaatacgactcactatag

SEQ ID NO: 120 - DNA, 48 bp, Artificial Sequence
  T7SD8M.F48 as Primer
taatacgactcactatagggttaactttaagaaggagatatacatatg

SEQ ID NO: 121 - DNA, 26 bp, Artificial Sequence
  T7g10.F26 as Primer
ctagtaatacgactcactatagggtt

SEQ ID NO: 122 - DNA, 20 bp, Artificial Sequence
  CGCStv.R20 as Primer
acatagttactgctggacgg

SEQ ID NO: 123 - DNA, 20 bp, Artificial Sequence
  NGC1Stv.R20 as Primer
acatagttactgctgaacgg

SEQ ID NO: 124 - DNA, 20 bp, Artificial Sequence
  NGC2Stv.R20 as Primer
acatagttactgctggacaa

SEQ ID NO: 125 - DNA, 38 bp, Artificial Sequence
  MYYYgcuDDuaaAS.R38 as Primer
cgaagcttagtcgtcagcgtagtagtacatgtttttct

SEQ ID NO: 126 - DNA, 38 bp, Artificial Sequence
  MYYYcguDDuaaAS.R38 as Primer
cgaagcttagtcgtcacggtagtagtacatgtttttct

SEQ ID NO: 127 - DNA, 38 bp, Artificial Sequence
  MYYYaauDDuaaAS.R38 as Primer
cgaagcttagtcgtcattgtagtagtacatgtttttct

SEQ ID NO: 128 - DNA, 38 bp, Artificial Sequence
  MYYYgauDDuaaAS.R38 as Primer
cgaagcttagtcgtcatcgtagtagtacatgtttttct

SEQ ID NO: 129 - DNA, 38 bp, Artificial Sequence
  MYYYuguDDuaaAS.R38 as Primer
cgaagcttagtcgtcacagtagtagtacatgtttttct

SEQ ID NO: 130 - DNA, 38 bp, Artificial Sequence
  MYYYcagDDuaaAS.R38 as Primer
cgaagcttagtcgtcctggtagtagtacatgtttttct

SEQ ID NO: 131 - DNA, 38 bp, Artificial Sequence
  MYYYgagDDuaaAS.R38 as Primer
cgaagcttagtcgtcctcgtagtagtacatgtttttct

SEQ ID NO: 132 - DNA, 38 bp, Artificial Sequence
  MYYYgguDDuaaAS.R38 as Primer
cgaagcttagtcgtcaccgtagtagtacatgtttttct

SEQ ID NO: 133 - DNA, 38 bp, Artificial Sequence
  MYYYcauDDuaaAS.R38 as Primer
cgaagcttagtcgtcatggtagtagtacatgtttttct

SEQ ID NO: 134 - DNA, 38 bp, Artificial Sequence
  MYYYauuDDuaaAS.R38 as Primer
cgaagcttagtcgtcaatgtagtagtacatgtttttct

SEQ ID NO: 135 - DNA, 38 bp, Artificial Sequence
  MYYYcuuDDuaaAS.R38 as Primer
cgaagcttagtcgtcaaggtagtagtacatgtttttct

SEQ ID NO: 136 - DNA, 38 bp, Artificial Sequence
  MYYYaagDDuaaAS.R38 as Primer
cgaagcttagtcgtccttgtagtagtacatgtttttct

SEQ ID NO: 137 - DNA, 38 bp, Artificial Sequence
  MYYYaugDDuaaAS.R38 as Primer
cgaagcttagtcgtccatgtagtagtacatgtttttct

SEQ ID NO: 138 - DNA, 38 bp, Artificial Sequence
  MYYYuuuDDuaaAS.R38 as Primer
cgaagcttagtcgtcaaagtagtagtacatgtttttct

SEQ ID NO: 139 - DNA, 38 bp, Artificial Sequence
  MYYYccuDDuaaAS.R38 as Primer
cgaagcttagtcgtcagggtagtagtacatgtttttct

SEQ ID NO: 140 - DNA, 38 bp, Artificial Sequence
  MYYYucuDDuaaAS.R38 as Primer
cgaagcttagtcgtcagagtagtagtacatgtttttct

SEQ ID NO: 141 - DNA, 38 bp, Artificial Sequence
  MYYYacuDDuaaAS.R38 as Primer
cgaagcttagtcgtcagtgtagtagtacatgtttttct

SEQ ID NO: 142 - DNA, 38 bp, Artificial Sequence
  MYYYuggDDuaaAS.R38 as Primer
cgaagcttagtcgtcccagtagtagtacatgtttttct

SEQ ID NO: 143 - DNA, 38 bp, Artificial Sequence
  MYYYuauDDuaaAS.R38 as Primer
cgaagcttagtcgtcatagtagtagtacatgtttttct

SEQ ID NO: 144 - DNA, 38 bp, Artificial Sequence
  MYYYguuDDuaaAS.R38 as Primer
cgaagcttagtcgtcaacgtagtagtacatgtttttct

SEQ ID NO: 145 - DNA, 55 bp, Artificial Sequence
  T7eSD6MYYY.F55 as Primer
taatacgactcactatagggttaactttaacaaggagaaaaacatgtactactac

SEQ ID NO: 146 - DNA, 24 bp, Artificial Sequence
  template DNA
  misc_feature (13)..(15): n is a, c, g, or t
atgtactactacnnngacgactaa

SEQ ID NO: 147 - protein, 7 aa, Artificial Sequence
  peptide translated from template DNA
  misc_feature (5)..(5): Xaa can be any naturally occurring amino acid
MYYYXDD

SEQ ID NO: 148 - DNA, 538 bp, Streptomyces avidinii
ctagtaatacgactcactatagggttaactttaagaaggagatatacatatggatccctctaaggactccaaggctcaggtttccgctgccgaggctggcattaccggcacttggtataaccagcttggctccacctttattgtcaccgctggcgctgatggtgctcttactggcacctatgagtctgccgttggcaatgctgagtcccgttatgttctcactggtcgctatgactccgctcctgctactgacggttctggtactgctcttggttggaccgttgcctggaagaacaattatcgtaacgcccattccgccaccacttggtctggtcagtatgttggtggtgctgaggctcgtatcaatactcagtggcttctcacctccggcactaccgaggctaacgcctggaagtctacccttgttggtcacgacactttcactaaggtcaagccctccgccgcctctatcgatgctgctaagaaggctggcgtcaataacggtaaccctcttgatgccgtccagcagtaactatgt

SEQ ID NO: 149 - DNA, 538 bp, Artificial Sequence
  NGC1.stv
ctagtaatacgactcactatagggttaactttaagaaggagatatacatatggatccccttaaggacctcaaggcccaggttcttgccgctgaggctggtattactggcacctggtacaaccagtctggcctcactttcatcgtcactgctggtgccgatggcgcttctaccggtacctatgagcttgccgttggcaatgctgagctccgctatgtctccaccggtcgttatgacctcgctcctgctactgacggtcttggcactgcttctggctggactgtcgcttggaagaacaactaccgcaatgcccatcttgccaccacttggcttggtcagtacgttggtggtgctgaggcccgtatcaacacccagtggtcctccactctcggcaccactgaggccaatgcctggaagctcacttccgttggtcacgacacctttaccaaggtcaagcctcttgctgccctcattgatgctgccaagaaggctggcgtcaacaatggcaatccctctgatgccgttcagcagtaactatgt

SEQ ID NO: 150 - DNA, 538 bp, Artificial Sequence
  NGC2.stv
ctagtaatacgactcactatagggttaactttaagaaggagatatacatatggatcctgccaaggatgctaagcttcaggttgcccttcttgagctcggcattactggcacctggtacaaccagtctggtgccaccttcatcgtcactcttggtcttgatggcctctctaccggcacctatgaggcccttgttggcaatctcgaggctcgctatgtctccactggtcgttatgacgctcttcccctcactgatggtgctggtaccctttctggctggactgtcctctggaagaacaactaccgcaatctccatgcccttaccacttgggctggtcagtacgttggcggtcttgagctccgtatcaacactcagtggtcctccactgctggtactactgagctcaatctctggaaggctacctccgttggtcacgacacctttaccaaggtcaagcctgcccttctcgccattgacctcctcaagaagcttggcgttaacaatggcaatccctctgaccttgtccagcagtaactatgt

SEQ ID NO: 151 - protein, 160 aa, Streptomyces avidinii
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 152 - protein, 160 aa, Artificial Sequence
  peptide sequence based on NGC1.stv
MDPLKDLKAQVLAAEAGITGTWYNQSGLTFIVTAGADGASTGTYELAVGNAELRYVSTGRYDLAPATDGLGTASGWTVAWKNNYRNAHLATTWLGQYVGGAEARINTQWSSTLGTTEANAWKLTSVGHDTFTKVKPLAALIDAAKKAGVNNGNPSDAVQQ

SEQ ID NO: 153 - protein, 160 aa, Artificial Sequence
  peptide sequence based on NGC2.stv
MDPAKDAKLQVALLELGITGTWYNQSGATFIVTLGLDGLSTGTYEALVGNLEARYVSTGRYDALPLTDGAGTLSGWTVLWKNNYRNLHALTTWAGQYVGGLELRINTQWSSTAGTTELNLWKATSVGHDTFTKVKPALLAIDLLKKLGVNNGNPSDLVQQ

(19) United States
(12) Patent Application Publication
(10) Pub. No.: US 2018/0298390 A1
(43) Pub. Date: Oct. 18, 2018

(54) Novel Genetic Code and Translation System for Producing Polypeptide, and Method for Producing Polypeptide

(71) Applicant: Hiroshi Murakami, Nagoya (JP)

(72) Inventors: Hiroshi MURAKAMI, Nagoya (JP); Takahiro ISHIZAWA, (US)

(21) Appl. No.: 15/764,015

(22) PCT Filed: Sep. 29, 2016
(86) PCT No.: PCT/JP2016/078921

§ 371 Date: Jul. 3, 2018

(30) Foreign Application Priority Data
Sep. 29, 2015 (JP) 2015-191730

(51) Int. Cl. C12N 15/52; C12P 21/02

(52) U.S. Cl. CPC C12N 15/52; C12Y 601/01011; C12N 2310/10; C12P 21/02

(57) ABSTRACT

The present invention provides a translation system for expressing a polypeptide having a predetermined amino acid sequence according to a code different from the universal genetic code, comprising a template nucleic acid having a sequence modified from a nucleic acid sequence encoding the polypeptide by interchanging codons encoding at least 2 amino acids selected from serine, leucine, and alanine; and at least either (i) or (ii):
(i) an aminoacyl-tRNA having an anticodon to a codon after interchange and to which an amino acid encoded by a codon before the interchange is bound;
(ii) a tRNA having an anticodon to a codon after interchange and recognized by an aminoacyl-tRNA synthetase corresponding to an amino acid encoded by a codon before the interchange, or a nucleic acid encoding the tRNA, the amino acids, and the aminoacyl-tRNA synthetase or a nucleic acid encoding the aminoacyl-tRNA synthetase.

### TECHNICAL FIELD

The present invention relates to a translation system for expressing a polypeptide using a new genetic code different from the universal genetic code.

### BACKGROUND ART

Proteins are synthesized from genes according to a genetic code. Since most living organisms use a common genetic code, living organisms can obtain genes from other living organisms by horizontal gene transfer, which allows the synthesis of useful proteins. In the engineering point of view, this common genetic code made it possible to introduce genes obtained from any living organism into Escherichia coli to produce useful proteins. Meanwhile, harmful genes may also transfer into and invade other living organisms and therefore it is feared, for example, exogenous genes introduced in genetically modified microorganisms flow into microorganisms in the natural world, or exogenous genes in genetically modified plants transfer to surrounding plants in the natural world.

Genes in living organisms having a genetic code greatly different from other living organisms do not encode proteins which are functional when transferred to other living organisms and therefore it is considered that such living organisms would be safe artificial living organisms. For example, if genetic codes of genetically modified microorganisms or plants are greatly different from the universal genetic codes, there is no risk of transferring modified genes as sense genes to living organisms in the natural world.

Moreover, in cell-free translation systems, use of a new genetic code different from the universal genetic code allows safe synthesis of proteins that require attention in handling the genes thereof.

Reported examples of use of a genetic code different from the universal genetic code include a study involving replacing all 314 TAG stop codons in the Escherichia coli genome with TAA stop codons (Non Patent Literature 1-3). This study also proposes making a TAG nonsense codon by removing the RF1 gene from this Escherichia coli genome and reassigning the TAG codon to an unnatural amino acid. However, genes based on this genetic code cannot be called as genes orthogonal to existing living organisms since such genes become easily translatable only by acquisition of a suppressor tRNA that reads the TAG codon by the living organism.

Meanwhile, the present inventors have succeed in designating Leu to a codon for Ala by replacing an anticodon loop of a tRNA^{Leu} with the anticodon loop same as that of a tRNA^{Ala} (Non Patent Literature 4).

### CITATION LIST

### Non Patent Literature

Non Patent Literature 1: F. J. Isaacs et al., Science Vol. 333, p. 348-353 (2011)
Non Patent Literature 2: M. J. Lajoie et al., Science Vol. 342, p. 357-360 (2013)
Non Patent Literature 3: D. J. Mandell et al., doi:10.1038/nature14121
Non Patent Literature 4: H. Murakami et al., Nature Structural & Molecular Biology Vol. 16, p. 353-358 (2009)

### SUMMARY OF INVENTION

### Technical Problem

An object of the present invention is to provide a translation system that can express polypeptide according to a genetic code different from the universal genetic code, and the like.

### Solution to Problem

To achieve the aforementioned object, the present inventors considered interchanging the relations between codons and amino acids to produce new genetic codes different from the universal genetic code by interchanging the anticodon loops of seryl-tRNA, leucyl-tRNA, and/or alanyl-tRNA, since it is well known that seryl-tRNA synthetase, leucyl-tRNA synthetase, and alanyl-tRNA synthetase do not recognize the anticodon portion of tRNA when catalyzing the aminoacylation of tRNAs.

More specifically, the present inventors interchanged codons for these amino acids in a gene encoding a protein and interchanged the relations between the anticodons and the amino acids in the aminoacyl-tRNAs correspondingly and succeeded in expressing polypeptides having predetermined amino acid sequences according to a new genetic code different from the universal genetic code, thereby completing the present invention.

Accordingly, the present invention relates to:

[1]

A translation system for expressing a polypeptide having a predetermined amino acid sequence according to a code different from the universal genetic code, comprising

a template nucleic acid having a sequence modified from the nucleic acid sequence encoding the polypeptide by interchanging codons encoding at least 2 amino acids selected from serine, leucine, and alanine; and

at least either (i) or (ii):

(i) an aminoacyl-tRNA having an anticodon to a codon after interchange and to which an amino acid encoded by a codon before the interchange is bound;

(ii) a tRNA having an anticodon to a codon after interchange and recognized by an aminoacyl-tRNA synthetase corresponding to an amino acid encoded by a codon before the interchange, or a nucleic acid encoding the tRNA, the amino acids, and the aminoacyl-tRNA synthetase or a nucleic acid encoding the aminoacyl-tRNA synthetase.

[2]

The translation system according to [1], wherein the template nucleic acid has a sequence modified from the nucleic acid sequence encoding the polypeptide by interchanging a codon encoding serine and a codon encoding leucine, and the translation system comprises

at least either (i) or (ii) and

at least either (iii) or (iv):

(i) an aminoacyl-tRNA having an anticodon to a codon encoding leucine and to which serine is bound;

(ii) a aminoacyl-tRNA having an anticodon to a codon encoding leucine and recognized by a seryl-tRNA synthetase or a nucleic acid encoding the tRNA, serine, and a seryl-tRNA synthetase or a nucleic acid encoding the seryl-tRNA synthetase;

(iii) an aminoacyl-tRNA having an anticodon to a codon encoding serine and to which leucine is bound;

(iv) a tRNA having an anticodon to a codon encoding serine and recognized by a leucyl-tRNA synthetase or a nucleic acid encoding the tRNA, leucine, and a leucyl-tRNA synthetase or a nucleic acid encoding the leucyl-tRNA synthetase.

[3]

The translation system according to [1], wherein the template nucleic acid has a sequence modified from the nucleic acid sequence encoding the polypeptide by interchanging a codon encoding serine and a codon encoding alanine, and the translation system comprises

at least either (i) or (ii) and

at least either (iii) or (iv):

(i) an aminoacyl-tRNA having an anticodon to a codon encoding alanine and to which serine is bound;

(ii) a tRNA having an anticodon to a codon encoding alanine and recognized by a seryl-tRNA synthetase or a nucleic acid encoding the tRNA, serine, and a seryl-tRNA synthetase or a nucleic acid encoding the seryl-tRNA synthetase;

(iii) an aminoacyl-tRNA having an anticodon to a codon encoding serine and to which alanine is bound;

(iv) a tRNA having an anticodon to a codon encoding serine and recognized by an alanyl-tRNA synthetase or a nucleic acid encoding the tRNA, alanine, and an alanyl-tRNA synthetase or a nucleic acid encoding the alanyl-tRNA synthetase.

[4]

The translation system according to [1], wherein the template nucleic acid has a sequence modified from the nucleic acid sequence encoding the polypeptide by interchanging a codon encoding leucine and a codon encoding alanine, and the translation system comprises

at least either (i) or (ii) and

at least either (iii) or (iv):

(i) an aminoacyl-tRNA having an anticodon to a codon encoding alanine and to which leucine is bound;

(ii) a tRNA having an anticodon to a codon encoding alanine and recognized by a leucyl-tRNA synthetase or a nucleic acid encoding the tRNA, leucine, and a leucyl-tRNA synthetase or a nucleic acid encoding the leucyl-tRNA synthetase;

(iii) an aminoacyl-tRNA having an anticodon to a codon encoding leucine and to which alanine is bound;

(iv) a tRNA having an anticodon to a codon encoding leucine and recognized by an alanyl-tRNA synthetase or a nucleic acid encoding the tRNA, alanine, and an alanyl-tRNA synthetase or a nucleic acid encoding the alanyl-tRNA synthetase.

[5]

The translation system according to [1], wherein the template nucleic acid has a sequence modified from the nucleic acid sequence encoding the polypeptide by exchanging a codon encoding serine for a codon encoding leucine, a codon encoding leucine for a codon encoding alanine, and a codon encoding alanine for a codon encoding serine, and the translation system comprises;

at least either (i) or (ii),

at least either (iii) or (iv), and

at least either (v) or (vi):

(i) an aminoacyl-tRNA having an anticodon to a codon encoding leucine and to which serine is bound;

(ii) a tRNA having an anticodon to a codon encoding leucine and recognized by a seryl-tRNA synthetase or a nucleic acid encoding the tRNA, serine, and a seryl-tRNA synthetase or a nucleic acid encoding the seryl-tRNA synthetase;

(iii) an aminoacyl-tRNA having an anticodon to a codon encoding alanine and to which leucine is bound;

(iv) a tRNA having an anticodon to a codon encoding alanine and recognized by a leucyl-tRNA synthetase or a nucleic acid encoding the tRNA, leucine, and a leucyl-tRNA synthetase or a nucleic acid encoding the leucyl-tRNA synthetase;

(v) an aminoacyl-tRNA having an anticodon to a codon encoding serine and to which alanine is bound;

(vi) a tRNA having an anticodon to a codon encoding serine and recognized by an alanyl-tRNA synthetase or a nucleic acid encoding the tRNA, alanine, and an alanyl-tRNA synthetase or a nucleic acid encoding the alanyl-tRNA synthetase.

[6]

The translation system according to [1], wherein the template nucleic acid has a sequence modified from the nucleic acid sequence encoding the polypeptide by exchanging a codon encoding serine for a codon encoding alanine, a codon encoding alanine for a codon encoding leucine, and a codon encoding leucine for a codon encoding serine, and the translation system comprises;

at least either (i) or (ii),

at least either (iii) or (iv), and

at least either (v) or (vi):

(i) an aminoacyl-tRNA having an anticodon to a codon encoding alanine and to which serine is bound;

(ii) a tRNA having an anticodon to a codon encoding alanine and recognized by a seryl-tRNA synthetase or a nucleic acid encoding the tRNA, serine, and a seryl-tRNA synthetase or a nucleic acid encoding the seryl-tRNA synthetase;

(iii) an aminoacyl-tRNA having an anticodon to a codon encoding leucine and to which alanine is bound;

(iv) a tRNA having an anticodon to a codon encoding leucine and recognized by an alanyl-tRNA synthetase or a nucleic acid encoding the tRNA, alanine, and an alanyl-tRNA synthetase or a nucleic acid encoding the alanyl-tRNA synthetase.

(v) an aminoacyl-tRNA having an anticodon to a codon encoding serine and to which leucine is bound;

(vi) a tRNA having an anticodon to a codon encoding serine and recognized by a leucyl-tRNA synthetase or a nucleic acid encoding the tRNA, leucine, and a leucyl-tRNA synthetase or a nucleic acid encoding the leucyl-tRNA synthetase.

[7]

A cell comprising the translation system according to any of [1] to [6].

[8]

A living organism comprising the translation system according to any of [1] to [6].

[9]

A method for producing a polypeptide having a predetermined amino acid sequence, comprising a step of:

expressing the polypeptide from the template nucleic acid in the translation system according to any of [1] to [6].

[10]

A method for producing a polypeptide having a predetermined amino acid sequence, comprising a step of:

expressing, in a translation system for expressing a polypeptide having a predetermined amino acid sequence according to a code different from the universal genetic code, the polypeptide from a template nucleic acid,

the translation system comprising:

the template nucleic acid having a sequence modified from a nucleic acid sequence encoding the polypeptide by interchanging codons encoding at least 2 amino acids selected from serine, leucine, and alanine; and

at least either (i) or (ii):

(i) an aminoacyl-tRNA having an anticodon to a codon after interchange and to which an amino acid encoded by a codon before the interchange is bound;

(ii) a tRNA having an anticodon to a codon after interchange and recognized by an aminoacyl-tRNA synthetase corresponding to an amino acid encoded by a codon before the interchange or a nucleic acid encoding the tRNA, the amino acid, and the aminoacyl-tRNA synthetase or a nucleic acid encoding the aminoacyl-tRNA synthetase.

[11]

A kit comprising

a translation system for expressing a polypeptide having a predetermined amino acid sequence according to a code different from the universal genetic code,

the translation system comprising:

a template nucleic acid having a sequence modified from a nucleic acid sequence encoding the polypeptide by interchanging codons encoding at least 2 amino acids selected from serine, leucine, and alanine; and

at least either (i) or (ii):

(i) an aminoacyl-tRNA having an anticodon to a codon after interchange and to which an amino acid encoded by a codon before the interchange is bound;

(ii) a tRNA having an anticodon to a codon after interchange and recognized by an aminoacyl-tRNA synthetase corresponding to an amino acid encoded by a codon before the interchange or a nucleic acid encoding the tRNA, the amino acid, and the aminoacyl-tRNA synthetase or a nucleic acid encoding the aminoacyl-tRNA synthetase.

### Advantageous Effects of Invention

With the translation system according to the present invention, polypeptides having predetermined amino acid sequences can be expressed according to a new genetic code different from the universal genetic code. Use of genes contained in such a translation system can prevent horizontal gene transfer of harmful genes, because such genes can be kept orthogonal to living organisms in the natural world. Polypeptides requiring attentions in handling their genes can be synthesized safely by applying the translation system according to the present invention to a cell-free translation system or a translation system using Escherichia coli,

Moreover, by applying the translation system according to the present invention to genetically modified living organisms such as genetically modified microorganisms or genetically modified plants, transmission of modified genes to living organisms in the natural world and expression of harmful polypeptides can be prevented.

### BRIEF DESCRIPTION OF DRAWINGS

FIG. 1 is a schematic diagram illustrating a genetic firewall by using a genetic code other than the universal genetic code. While functional proteins are expressed from genes encoded by the universal genetic code in natural translation systems, non-functional proteins are expressed from a translation system using another genetic code (Firewall A). Meanwhile, in the translation system using another genetic code, functional proteins are expressed from genes encoded by another genetic code and, in the natural translation system, only non-functional proteins are expressed (Firewall B).

FIG. 2 illustrates the universal genetic code and codon-shuffled genetic codes. (A) illustrates the universal genetic code. tRNA^{Ala}, tRNA^{Leu}, and tRNA^{Ser} have the original anticodon loops. (B) illustrates codon-shuffled genetic code 1. The CUU and CUC codons are assigned to Ser and the UCU and UCC codons are assigned to Leu and also chimeric tRNA^{Leu} and chimeric tRNA^{Ser} in which the anticodon loops are interchanged are used. (C) illustrates codon-shuffled genetic code 2. The GCU and GCC codons are assigned to Ser, the CUU and CUC codons are assigned to Ala, and the UCU and UCC codons are assigned to Leu and also chimeric tRNA^{Ala}, chimeric tRNA^{Leu}, and chimeric tRNA^{Ser} in which the anticodon loops are interchanged are used.

FIG. 3 illustrates orthogonal expression of streptavidin using the universal genetic code (Can), codon-shuffled genetic code 1 (CS1), and codon-shuffled genetic code 2 (CS2). The upper panel illustrates the result of the Tricine-SDS-PAGE analysis. Proteins were labelled with [¹⁴C]-Asp Asp and detected by autoradiography. The biotin binding activity was examined by the dot blot assay using biotin conjugated horseradish peroxidase (biotin-HRP) (lower panel). Lane 1, dot 1: the native tRNAs and the universal streptavidin gene were used. Lane 2, dot 2: the native tRNAs and no universal streptavidin gene were used. Lane 3, dot 3: the in vitro transcribed universal tRNA set and the universal streptavidin gene were used. Lane 4, dot 4: the in vitro transcribed universal tRNA set and the codon-shuffled 1 streptavidin gene were used. Lane 5, dot 5: the in vitro transcribed universal tRNA set and the codon-shuffled 2 streptavidin gene were used. Lane 6, dot 6: the in vitro transcribed codon-shuffled 1 tRNA set and the universal streptavidin gene were used. Lane 7, dot 7: the in vitro transcribed codon-shuffled 1 tRNA set and the codon-shuffled 1 streptavidin gene were used. Lane 8, dot 8: the in vitro transcribed codon-shuffled 1 tRNA set and the codon-shuffled 2 streptavidin gene were used. Lane 9, dot 9: the in vitro transcribed codon-shuffled 2 tRNA set and the universal streptavidin gene were used. Lane 10, dot 10: the in vitro transcribed codon-shuffled 2 tRNA set and the codon-shuffled 1 streptavidin gene were used. Lane 11, dot 11: the in vitro transcribed codon-shuffled 2 tRNA set and the codon-shuffled 2 streptavidin gene were used. The error bars indicate the standard deviation calculated from 3 experiments.

FIG. 4 illustrates the tRNA activity analysis by the peptide expression assay. (A) illustrates sequences of the template DNA (upper line) and the expressed peptide (lower line). (B) illustrates the result of the Tricine-SDS-PAGE analysis of peptides expressed in the translation system using an in vitro transcribed tRNA set (tRNA^{fMet}, tRNA^{Asp}, tRNA^{Tyr}, and tRNA^{Xaa}). The peptides were labelled with [¹⁴C]-Asp and detected by autoradiography. The expression level of each peptide was confirmed from the band in gel. (C) illustrates the result of the Tricine-SDS-PAGE analysis and the autoradiography analysis of the peptides expressed in the translation system using native tRNAs.

FIG. 5 illustrates the result of the MALDI-TOF-MS analysis of peptides expressed with native tRNAs. Asp was used instead of [¹⁴C]-Asp. Asp. The amino acids and the numbers in the parenthesis indicate the Xaas and the Lane numbers in FIG. 4, respectively.

FIG. 6 illustrates the result of the MALDI-TOF-MS analysis of peptides expressed with in vitro transcribed tRNA^{fMet}, tRNA^{Asp}, tRNA^{Tyr}, and tRNA^{Xaa}. Asp was used instead of [¹⁴C]-Asp. Asp. The amino acids and the numbers in the parenthesis indicate the Xaas and the Lane numbers in FIG. 4, respectively.

FIG. 7 illustrates the result of analysis on accuracy of translation with the natural tRNA set and in vitro transcribed tRNA sets. (A) illustrates sequences of the template DNA (upper line) and the expressed peptide (lower line). Since the peptide is translated in the absence of Xaa corresponding to the NNN codon, the decoding error activity at the NNN codon is illustrated. The expressed peptide was labelled with [¹⁴C]-Asp. Asp. (B) illustrates the result of the SDS-PAGE analysis when the peptide was expressed with native tRNA. (C) illustrates the result of the SDS-PAGE analysis when the peptide was expressed with the in vitro transcribed universal tRNA set (tRNA No. 0-20 in Table 5).

FIG. 8 illustrates the alignment of streptavidin gene sequences. The upper line indicates the sequence according to the universal genetic code, the middle line indicates the sequence according to codon-shuffled genetic code 1, and the lower line is the sequence according to codon-shuffled genetic code 2. The genes contain common T7 promoter and Shine-Dalgarno (SD) sequences in their 5′-untranslated regions.

FIG. 9 illustrates the alignment of protein sequences. The upper line indicates the sequence according to the universal genetic code, the middle line indicates the sequence according to codon-shuffled genetic code 1, and the lower line is the sequence according to codon-shuffled genetic code 2. The asterisks indicate preserved amino acid residues.

### DESCRIPTION OF EMBODIMENTS

The outline of the translation system according to the present invention is described. The translation system according to the present invention is a translation system to express a polypeptide having a predetermined amino acid sequence according to a code different from the universal genetic code and comprises a template nucleic acid having a sequence modified from a nucleic acid sequence encoding the polypeptide by interchanging codons encoding at least 2 amino acids selected from serine, leucine, and alanine; and

aminoacyl-tRNAs having anticodons to codons after the interchange and to which the amino acids encoded by the codons before the interchange are bound.

As used herein, the “polypeptide” refers to a molecule composed of amino acids linked by the peptide bond and is not particularly limited as long as the number of the amino acids thereof is 2 or more. The polypeptides include those called as proteins and peptides. As used herein, the “polypeptide having a predetermined amino acid sequence” refers to a polypeptide whose amino acid sequence is known and that is to be expressed in the translation system according to the present invention.

As used herein, “to express polypeptide” is, unless otherwise specified, used as a term including both transcription, by which mRNA is synthesized based on the sequence of DNA, and translation, by which polypeptide is synthesized based on the sequence of mRNA.

As used herein, the term “aminoacyl-tRNAs having anticodons to codons after the interchange and to which the amino acids encoded by the codons before the interchange are bound” means aminoacyl-tRNAs, for example when codons for serine are interchanged with codons for leucine, aminoacyl-tRNAs having anticodons to the codons for leucine in the universal genetic code after the interchange and to which serine is bound, which is the amino acid encoded by the codons before the interchange. By using such aminoacyl-tRNAs, a polypeptide having the original amino acid sequence is expressed based on the template nucleic acid, in which the codons have been interchanged. Therefore, in this translation system, the relation between the codons and the amino acids will follow the genetic code different from the universal genetic code.

Moreover, the translation system according to the present invention may comprise, instead of “aminoacyl-tRNAs having anticodons to codons after the interchange and to which the amino acids encoded by the codons before the interchange are bound” tRNAs or nucleic acids encoding the tRNAs having anticodons to codons after the interchange and recognized by aminoacyl-tRNA synthetases corresponding to the amino acids encoded by the codons before the interchange or nucleic acids encoding the tRNAs, the amino acids, and the aminoacyl-tRNA synthetases or nucleic acids encoding the aminoacyl-tRNA synthetases. With such a configuration, “aminoacyl-tRNAs having anticodons to codons after the interchange and to which the amino acids encoded by the codons before the interchange are bound” are produced by the aminoacyl-tRNA synthetases in the translation system.

As used herein, the tRNA means transfer RNA. As used herein, the seryl-tRNA (tRNA^{Ser}), the leucyl-tRNA (tRNA^{Leu}), and the alanyl-tRNA (tRNA^{Ala}) mean the natural tRNAs that transport serine, leucine, and alanine, respectively. Therefore, they have the anticodons to the codons for serine, leucine, and alanine, respectively.

As used herein, the “codon” means a sequence that specifies one amino acid, and consists of 3 nucleotides selected from T (thymine), C (cytosine), A (adenine), and G (guanine), in the case of DNA. In the codons in mRNA, T (thymine) is replaced with U (uracil).

As used herein, the “nucleic acid sequence encoding X” means a nucleic acid sequence encoding the amino acid sequence of X according to the universal genetic code. As used herein, the “nucleic acid” may be DNA or RNA and may include chimera of DNA and RNA and artificial nucleic acids, as long as an object of the present invention is achieved.

As used herein, the “universal genetic code” refers to the following genetic code in the case of DNA and genes of most species of organisms on the earth encode amino acid sequences according to this code. In the case of RNA, T is replaced with U. Hereinafter, the codons are described basically as DNA sequences, but in the case of RNA, T should be replaced with U.

As used herein, genes to be decoded according to the universal genetic code may be referred to as universal genes and tRNAs that decode mRNA according to the universal genetic code may be referred to as universal tRNAs.

TABLE 1

| | T | | C | | A | | G | | |
|---|---|---|---|---|---|---|---|---|---|
| T | TTT | Phe | TCT | Ser | TAT | Tyr | TGT | Cys | T |
| | TTC | | TCC | | TAC | | TGC | | C |
| | TTA | Leu | TCA | | TAA | STOP | TGA | STOP | A |
| | TTG | | TCG | | TAG | | TGG | Trp | G |
| C | CTT | Leu | CCT | Pro | CAT | His | CGT | Arg | T |
| | CTC | | CCC | | CAC | | CGC | | C |
| | CTA | | CCA | | CAA | Gln | CGA | | A |
| | CTG | | CCG | | CAG | | CGG | | G |
| A | ATT | Ile | ACT | Thr | AAT | Asn | AGT | Ser | T |
| | ATC | | ACC | | AAC | | AGC | | C |
| | ATA | | ACA | | AAA | Lys | AGA | Arg | A |
| | ATG | Met | ACG | | AAG | | AGG | | G |
| G | GTT | Val | GCT | Ala | GAT | Asp | GGT | Gly | T |
| | GTC | | GCC | | GAC | | GGC | | C |
| | GTA | | GCA | | GAA | Glu | GGA | | A |
| | GTG | | GCG | | GAG | | GGG | | G |

Hereinafter, the first aspect wherein codons for serine and leucine are interchanged; the second aspect wherein codons for serine and alanine are interchanged; the third aspect wherein codons for leucine and alanine are interchanged; the fourth aspect wherein a codon for serine is exchanged with a codon for leucine, a codon for leucine is exchanged with a codon for alanine, and a codon for alanine is exchanged with a codon for serine; and the fifth aspect wherein a codon for serine is exchanged with a codon for alanine, a codon for alanine is exchanged with a codon for leucine, and a codon for leucine is exchanged with a codon for serine will each be described in detail.

The first aspect of the translation system according to the present invention comprises a template nucleic acid having a sequence modified from a nucleic acid sequence encoding a polypeptide having a predetermined amino acid sequence by interchanging codons for serine and leucine.

In the universal genetic code, serine is encoded by TCT, TCC, TCA, TCG, AGT, or AGC. Meanwhile, leucine is encoded by TTA, TTG, CTT, CTC, CTA, or CTG. In a sequence modified from a nucleic acid sequence encoding a predetermined protein by interchanging codons for serine and leucine, TCT, TCC, TCA, TCG, AGT, or AGC is replaced with any of TTA, TTG, CTT, CTC, CTA, and CTG and TTA, TTG, CTT, CTC, CTA, or CTG is replaced with any of TCT, TCC, TCA, TCG, AGT, and AGC in the nucleic acid sequence encoding the protein.

Such a nucleic acid can be synthesized by a usual method of nucleic acid synthesis.

The first aspect of the translation system according to the present invention comprises an aminoacyl-tRNA comprising a tRNA having an anticodon to a codon for leucine and serine bound to the tRNA. With such an aminoacyl-tRNA, a codon for leucine, that is to say, TTA, TTG, CTT, CTC, CTA, or CTG is translated to serine. Since a codon for serine and a codon for leucine are interchanged in the nucleic acid sequence of the first aspect as described above, the positions where the codon for serine was present before the interchange are thereby translated to serine.

In the case where codons for serine and leucine are interchanged in the present invention, it is not necessary that all codons are interchanged. For example, in a sequence in which some codons for leucine and serine are interchanged, AGT and AGC are replaced with TTA or TTG and TTA and TTG are replaced with any of AGT and AGC in a nucleic acid sequence encoding the protein. In this case, the correct protein can be translated by interchanging the anticodons of tRNA^{Ser} and tRNA^{Leu} corresponding to these codons. This is true for the case where codons for serine and alanine, alanine and leucine, or serine, alanine, and leucine are interchanged.

The first aspect of the translation system according to the present invention may comprise, instead of an aminoacyl-tRNA comprising a tRNA having an anticodon to a codon for leucine and serine bound to the tRNA, a tRNA having an anticodon to a codon for leucine and is recognized by a seryl-tRNA synthetase or a nucleic acid encoding the tRNA, serine, and a seryl-tRNA synthetase or a nucleic acid encoding the seryl-tRNA synthetase. In this way, a tRNA having an anticodon to a codon for leucine and recognized by a seryl-tRNA synthetase and the seryl-tRNA synthetase are expressed in the translation system and the tRNA and serine are bound by the reaction catalyzed by the enzyme to produce an aminoacyl-tRNA comprising a tRNA having an anticodon to a codon for leucine and serine bound to the tRNA.

The “tRNA having an anticodon to a codon for leucine and recognized by a seryl-tRNA synthetase” can be produced by modifying the sequence of the anticodon arm of the tRNA^{Ser} into a sequence containing an anticodon to a codon for leucine. Only the sequence of the anticodon part in the anticodon arm may be modified or a part including a neighboring sequence may also be modified. For example, the anticodon loop of the tRNA^{Ser} may be exchanged for the anticodon loop of the tRNA^{Leu} or the anticodon arm of the tRNA^{Ser} may be exchanged for the anticodon arm of the tRNA^{Leu}. Since the seryl-tRNA synthetase, which binds serine to tRNA^{Ser}, recognizes parts other than the anticodon arm of tRNA^{Ser}, it can bind serine to modified tRNA^{Ser}s in which the sequence of the anticodon arm is modified.

The first aspect of the translation system according to the present invention comprises an aminoacyl-tRNA comprising a tRNA having an anticodon to a codon for serine and leucine bound to the tRNA. With such an aminoacyl-tRNA, a codon for serine, that is, TCT, TCC, TCA, TCG, AGT, or AGC is translated to leucine. Since a codon for serine and a codon for leucine are interchanged in the nucleic acid sequence of the first aspect as described above, the positions where the codon for leucine was present before the interchange are thereby translated to leucine.

The first aspect of the translation system according to the present invention may comprise, instead of an aminoacyl-tRNA comprising a tRNA having an anticodon to a codon for serine and leucine bound to the tRNA, a tRNA having an anticodon to a codon for serine and recognized by a leucyl-tRNA synthetase or a nucleic acid encoding the tRNA and a leucyl-tRNA synthetase or a nucleic acid encoding the leucyl-tRNA synthetase. In this way, a tRNA having an anticodon to a codon for serine and recognized by a leucyl-tRNA synthetase and the leucyl-tRNA synthetase are expressed in the translation system and the tRNA and leucine are bound by the reaction catalyzed by the enzyme to produce an aminoacyl-tRNA comprising a tRNA having an anticodon to a codon for serine and leucine bound to the tRNA.

The “tRNA having an anticodon to a codon for serine and recognized by a leucyl-tRNA synthetase” can be produced by modifying the sequence of the anticodon arm of the tRNA^{Leu} into a sequence containing an anticodon to a codon for serine. Only the sequence of the anticodon part in the anticodon arm and a neighboring sequence may be modified. For example, the anticodon loop of the tRNA^{Leu} may be exchanged for the anticodon loop of the tRNA^{Ser} or the anticodon arm of the tRNA^{Leu} may be exchanged for the anticodon arm of the tRNA^{Ser}. Since the leucyl-tRNA synthetase, which binds leucine to tRNA^{Leu}, also recognizes parts other than the anticodon arm of tRNA^{Leu}, it can bind leucine to modified tRNA^{Leu}s in which the sequence of the anticodon arm is modified.

The second aspect of the translation system according to the present invention comprises a template nucleic acid having a sequence modified from a nucleic acid sequence encoding a polypeptide having a predetermined amino acid sequence by interchanging codons for serine and alanine. In the universal genetic code, serine is encoded by TCT, TCC, TCA, TCG, AGT, or AGC and alanine is encoded by GCT, GCC, GCA, or GCG. Therefore, in a sequence modified from a nucleic acid sequence encoding a predetermined protein by interchanging codons for serine and alanine, TCT, TCC, TCA, TCG, AGT, and AGC are replaced with any of GCT, GCC, GCA, and GCG and GCT, GCC, GCA, and GCG are replaced with any of TCT, TCC, TCA, TCG, AGT, and AGC in the nucleic acid sequence encoding the protein.

The second aspect of the translation system according to the present invention comprises an aminoacyl-tRNA comprising a tRNA having an anticodon to a codon for alanine and serine bound to the tRNA. With such an aminoacyl-tRNA, a codon for alanine is translated to serine. Since a codon for serine and a codon for alanine are interchanged in the nucleic acid sequence of the second aspect as described above, the positions where the codon for serine was present before the interchange are thereby translated to serine.

The second aspect of the translation system according to the present invention may comprise, instead of an aminoacyl-tRNA comprising a tRNA having an anticodon to a codon for alanine and serine bound to the tRNA, a tRNA having an anticodon to a codon for alanine and recognized by a seryl-tRNA synthetase or a nucleic acid encoding the tRNA, serine, and the seryl-tRNA synthetase or a nucleic acid encoding the seryl-tRNA synthetase. In this way, a tRNA having an anticodon to a codon for alanine and is recognized by a seryl-tRNA synthetase and the seryl-tRNA synthetase are expressed in the translation system and the tRNA and serine are bound by the reaction catalyzed by the enzyme to produce an aminoacyl-tRNA comprising a tRNA having an anticodon to a codon for alanine and serine bound to the tRNA.

The “tRNA having an anticodon to a codon for alanine and is recognized by a seryl-tRNA synthetase” can be produced by modifying the sequence of the anticodon arm of the tRNA^{Ser} into a sequence containing an anticodon to a codon for alanine. Only the sequence of the anticodon part in the anticodon arm may be modified or a part including a neighboring sequence may also be modified. For example, the anticodon loop of the tRNA^{Ser} may be exchanged for the anticodon loop of the tRNA^{Ala} or the anticodon arm of the tRNA^{Ser} may be exchanged for the anticodon arm of the tRNA^{Ala}.

The second aspect of the translation system according to the present invention comprises an aminoacyl-tRNA comprising a tRNA having an anticodon to a codon for serine and alanine bound to the tRNA. With such an aminoacyl-tRNA, a codon for serine is translated to alanine. Since a codon for serine and a codon for alanine are interchanged in the nucleic acid sequence of the second aspect as described above, the positions where the codon for alanine was present before the interchange are thereby translated to alanine.

The second aspect of the translation system according to the present invention may comprise, instead of an aminoacyl-tRNA comprising a tRNA having an anticodon to a codon for serine and alanine bound to the tRNA, a tRNA having an anticodon to a codon for serine and recognized by an alanyl-tRNA synthetase or a nucleic acid encoding the tRNA and the alanyl-tRNA synthetase or a nucleic acid encoding the alanyl-tRNA synthetase. In this way, a tRNA having an anticodon to a codon for serine and recognized by an alanyl-tRNA synthetase and the alanyl-tRNA synthetase are expressed in the translation system and the tRNA and alanine are bound by the reaction catalyzed by the enzyme to produce an aminoacyl-tRNA comprising a tRNA having an anticodon to a codon for serine and alanine bound to the tRNA.

The “tRNA having an anticodon to a codon for serine and recognized by an alanyl-tRNA synthetase” can be produced by modifying the sequence of the anticodon arm of the tRNA^{Ala} into a sequence containing an anticodon to a codon for serine. Only the sequence of the anticodon part in the anticodon arm and a neighboring sequence may be modified. For example, the anticodon loop of the tRNA^{Ala} may be exchanged for the anticodon loop of the tRNA^{Ser} or the anticodon arm of the tRNA^{Ala} may be exchanged for the anticodon arm of the tRNA^{Ser}. Since the alanyl-tRNA synthetase, which binds alanine to tRNA^{Ala}, also recognizes parts other than the anticodon arm of tRNA^{Ala}, it can bind alanine to modified tRNA^{Ala} in which the sequence of the anticodon arm is modified.

The third aspect of the translation system according to the present invention comprises a template nucleic acid having a sequence modified from a nucleic acid sequence encoding a polypeptide having a predetermined amino acid sequence by interchanging codons for leucine and alanine. In the universal genetic code, leucine is encoded by TTA, TTG, CTT, CTC, CTA, or CTG and alanine is encoded by GCT, GCC, GCA, or GCG. Therefore, in a sequence modified from a nucleic acid sequence encoding a predetermined protein by interchanging codons for leucine and alanine, TTA, TTG, CTT, CTC, CTA, and CTG are replaced with any of GCT, GCC, GCA, and GCG and GCT, GCC, GCA, and GCG are replaced with any of TTA, TTG, CTT, CTC, CTA, and CTG in the nucleic acid sequence encoding the protein.

The third aspect of the translation system according to the present invention comprises an aminoacyl-tRNA comprising a tRNA having an anticodon to a codon for alanine and leucine bound to the tRNA. With such an aminoacyl-tRNA, a codon for alanine is translated to leucine. Since a codon for alanine and a codon for leucine are interchanged in the nucleic acid sequence of the third aspect as described above, the positions where the codon for leucine was present before the interchange are thereby translated to leucine.

The third aspect of the translation system according to the present invention may comprise, instead of an aminoacyl-tRNA comprising a tRNA having an anticodon to a codon for alanine and leucine bound to the tRNA, a tRNA having an anticodon to a codon for alanine and recognized by a leucyl-tRNA synthetase or a nucleic acid encoding the tRNA, leucine, and the leucyl-tRNA synthetase or a nucleic acid encoding the leucyl-tRNA synthetase. In this way, a tRNA having an anticodon to a codon for alanine and recognized by a leucyl-tRNA synthetase and the leucyl-tRNA synthetase are expressed in the translation system and the tRNA and leucine are bound by the reaction catalyzed by the enzyme to produce an aminoacyl-tRNA comprising a tRNA having an anticodon to a codon for alanine and leucine bound to the tRNA.

The “tRNA having an anticodon to a codon for alanine and recognized by a leucyl-tRNA synthetase” can be produced by modifying the sequence of the anticodon arm of the tRNA^{Leu} into a sequence containing an anticodon to a codon for alanine. Only the sequence of the anticodon part in the anticodon arm may be modified or a part including a neighboring sequence may also be modified.

The third aspect of the translation system according to the present invention comprises an aminoacyl-tRNA comprising a tRNA having an anticodon to a codon for leucine and alanine bound to the tRNA. With such an aminoacyl-tRNA, a codon for leucine is translated to alanine. Since a codon for leucine and a codon for alanine are interchanged in the nucleic acid sequence of the third aspect as described above, the positions where the codon for alanine was present before the interchange are thereby translated to alanine.

The third aspect of the translation system according to the present invention may comprise, instead of an aminoacyl-tRNA comprising a tRNA having an anticodon to a codon for leucine and alanine bound to the tRNA, a tRNA having an anticodon to a codon for leucine and recognized by an alanyl-tRNA synthetase or a nucleic acid encoding the tRNA, alanine, and the alanyl-tRNA synthetase or a nucleic acid encoding the alanyl-tRNA synthetase. In this way, a tRNA having an anticodon to a codon for leucine and recognized by an alanyl-tRNA synthetase and the alanyl-tRNA synthetase are expressed in the translation system and the tRNA and alanine are bound by the reaction catalyzed by the enzyme to produce an aminoacyl-tRNA comprising a tRNA having an anticodon to a codon for leucine and alanine bound to the tRNA.

The “tRNA having an anticodon to a codon for leucine and recognized by an alanyl-tRNA synthetase” can be produced by modifying the sequence of the anticodon arm of the tRNA^{Ala} into a sequence containing an anticodon to a codon for leucine. Only the sequence of the anticodon part in the anticodon arm and a neighboring sequence may be modified. For example, the anticodon loop of the tRNA^{Ala} may be exchanged for the anticodon loop of the tRNA^{Leu} or the anticodon arm of the tRNA^{Ala} may be exchanged for the anticodon arm of the tRNA^{Leu}.

The fourth aspect of the “translation system for producing a polypeptide having a predetermined amino acid sequence” according to the present invention comprises a template nucleic acid having a sequence modified from a nucleic acid sequence encoding the predetermined protein by exchanging a codon for serine with a codon for leucine, a codon for leucine with a codon for alanine, and a codon for alanine with a codon for serine. In the universal genetic code, serine is encoded by TCT, TCC, TCA, TCG, AGT, or AGC, leucine is encoded by TTA, TTG, CTT, CTC, CTA, or CTG, and alanine is encoded by GCT, GCC, GCA, or GCG. Therefore, in a sequence modified from a nucleic acid sequence encoding the predetermined protein by exchanging a codon for serine with a codon for leucine, a codon for leucine with a codon for alanine, and a codon for alanine with a codon for serine, TCT, TCC, TCA, TCG, AGT, and AGC are replaced with any of TTA, TTG, CTT, CTC, CTA, and CTG; TTA, TTG, CTT, CTC, CTA, and CTG are replaced with any of GCT, GCC, GCA, and GCG; GCT, GCC, GCA, and GCG are replaced with any of TCT, TCC, TCA, TCG, AGT, and AGC in the nucleic acid sequence encoding the protein.

The fourth aspect of the translation system according to the present invention comprises an aminoacyl-tRNA comprising a tRNA having an anticodon to a codon for leucine and serine bound to the tRNA; an aminoacyl-tRNA comprising a tRNA having an anticodon to a codon for alanine and leucine bound to the tRNA; and an aminoacyl-tRNA comprising a tRNA having an anticodon to a codon for serine and alanine bound to the tRNA. In this way, a codon for leucine is translated to serine, a codon for alanine is translated to leucine, and a codon for serine is translated to alanine. Since a codon for serine is exchanged with a codon for leucine, a codon for leucine is exchanged with a codon for alanine, and a codon for alanine is exchanged with a codon for serine in the fourth aspect as described above, the positions where the codon for serine was present before exchange are translated to serine, the positions where the codon for leucine was present are translated to leucine, and the positions where the codon for alanine was present are translated to alanine in this way to produce a predetermined polypeptide having an original amino acid sequence from the template nucleic acid by translation.

The forth aspect of the translation system according to the present invention may comprise, instead of an aminoacyl-tRNA comprising a tRNA having an anticodon to a codon for leucine and serine bound to the tRNA, a tRNA having an anticodon to a codon for leucine and recognized by a seryl-tRNA synthetase or a nucleic acid encoding the tRNA, serine, and a seryl-tRNA synthetase or a nucleic acid encoding the seryl-tRNA synthetase. In this way, a tRNA having an anticodon to a codon for leucine and recognized by a seryl-tRNA synthetase and the seryl-tRNA synthetase are expressed in the translation system and the tRNA and serine are bound by the reaction catalyzed by the enzyme to produce an aminoacyl-tRNA comprising a tRNA having an anticodon to a codon for leucine and serine bound to the tRNA.

The fourth aspect of the translation system according to the present invention may comprise, instead of an aminoacyl-tRNA comprising a tRNA having an anticodon to a codon for alanine and leucine bound to the tRNA, a tRNA having an anticodon to a codon for alanine and recognized by a leucyl-tRNA synthetase or a nucleic acid encoding the tRNA, leucine, and the leucyl-tRNA synthetase or a nucleic acid encoding the leucyl-tRNA synthetase. In this way, a tRNA having an anticodon to a codon for alanine and recognized by a leucyl-tRNA synthetase and the leucyl-tRNA synthetase are expressed in the translation system and the tRNA and leucine are bound by the reaction catalyzed by the enzyme to produce an aminoacyl-tRNA comprising a tRNA having an anticodon to a codon for alanine and leucine bound to the tRNA.

The fourth aspect of the translation system according to the present invention may comprise, instead of an aminoacyl-tRNA comprising a tRNA having an anticodon to a codon for serine and alanine bound to the tRNA, a tRNA having an anticodon to a codon for serine and recognized by an alanyl-tRNA synthetase or a nucleic acid encoding the tRNA and the alanyl-tRNA synthetase or a nucleic acid encoding the alanyl-tRNA synthetase. In this way, a tRNA having an anticodon to a codon for serine and recognized by an alanyl-tRNA synthetase and the alanyl-tRNA synthetase are expressed in the translation system and the tRNA and alanine are bound by the reaction catalyzed by the enzyme to produce an aminoacyl-tRNA comprising a tRNA having an anticodon to a codon for serine and alanine bound to the tRNA.

The fifth aspect of the “translation system for producing a polypeptide having a predetermined amino acid sequence” according to the present invention comprises a template nucleic acid having a sequence modified from a nucleic acid sequence encoding the predetermined protein by exchanging a codon for serine with a codon for alanine, a codon for alanine with a codon for leucine, and a codon for leucine with a codon for serine. In the universal genetic code, serine is encoded by TCT, TCC, TCA, TCG, AGT, or AGC, alanine is encoded by GCT, GCC, GCA, or GCG, and leucine is encoded by TTA, TTG, CTT, CTC, CTA, or CTG. Therefore, in a sequence modified from a nucleic acid sequence encoding the predetermined protein by exchanging a codon for serine with a codon for alanine, a codon for alanine with a codon for leucine, and a codon for leucine with a codon for serine, TCT, TCC, TCA, TCG, AGT, and AGC are replaced with any of GCT, GCC, GCA, and GCG; GCT, GCC, GCA, and GCG are replaced with any of TTA, TTG, CTT, CTC, CTA, and CTG; TTA, TTG, CTT, CTC, CTA, and CTG are replaced with any of TCT, TCC, TCA, TCG, AGT, and AGC in the nucleic acid sequence encoding the protein.

The fifth aspect of the translation system according to the present invention comprises an aminoacyl-tRNA comprising a tRNA having an anticodon to a codon for alanine and serine bound to the tRNA; an aminoacyl-tRNA comprising a tRNA having an anticodon to a codon for leucine and alanine bound to the tRNA; and an aminoacyl-tRNA comprising a tRNA having an anticodon to a codon for serine and leucine bound to the tRNA. In this way, a codon for alanine is translated to serine, a codon for leucine is translated to alanine, and a codon for serine is translated to leucine. Since a codon for serine is exchanged with a codon for alanine, a codon for alanine is exchanged with a codon for leucine, and a codon for leucine is exchanged with a codon for serine in the fifth aspect as described above, the positions where the codon for serine was present before exchange are translated to serine, the positions where the codon for leucine was present are translated to leucine, and the positions where the codon for alanine was present are translated to alanine in this way to produce a predetermined polypeptide having an original amino acid sequence from the template nucleic acid by translation.

The fifth aspect of the translation system according to the present invention may comprise, instead of an aminoacyl-tRNA comprising a tRNA having an anticodon to a codon for alanine and serine bound to the tRNA, a tRNA having an anticodon to a codon for alanine and recognized by a seryl-tRNA synthetase or a nucleic acid encoding the tRNA, serine, and the seryl-tRNA synthetase or a nucleic acid encoding the seryl-tRNA synthetase. In this way, a tRNA having an anticodon to a codon for alanine and is recognized by a seryl-tRNA synthetase and the seryl-tRNA synthetase are expressed in the translation system and the tRNA and serine are bound by the reaction catalyzed by the enzyme to produce an aminoacyl-tRNA comprising a tRNA having an anticodon to a codon for alanine and serine bound to the tRNA.

The fifth aspect of the translation system according to the present invention may comprise, instead of an aminoacyl-tRNA comprising a tRNA having an anticodon to a codon for leucine and alanine bound to the tRNA, a tRNA having an anticodon to a codon for leucine and recognized by an alanyl-tRNA synthetase or a nucleic acid encoding the tRNA, alanine, and the alanyl-tRNA synthetase or a nucleic acid encoding the alanyl-tRNA synthetase. In this way, a tRNA having an anticodon to a codon for leucine and is recognized by an alanyl-tRNA synthetase and the alanyl-tRNA synthetase are expressed in the translation system and the tRNA and alanine are bound by the reaction catalyzed by the enzyme to produce an aminoacyl-tRNA comprising a tRNA having an anticodon to a codon for leucine and alanine bound to the tRNA.

The fifth aspect of the translation system according to the present invention may comprise, instead of an aminoacyl-tRNA comprising a tRNA having an anticodon to a codon for serine and leucine bound to the tRNA, a tRNA having an anticodon to a codon for serine and recognized by a leucyl-tRNA synthetase or a nucleic acid encoding the tRNA, leucine, and the leucyl-tRNA synthetase or a nucleic acid encoding the leucyl-tRNA synthetase. In this way, a tRNA having an anticodon to a codon for serine and recognized by a leucyl-tRNA synthetase and the leucyl-tRNA synthetase are expressed in the translation system and the tRNA and leucine are bound by the reaction catalyzed by the enzyme to produce an aminoacyl-tRNA comprising a tRNA having an anticodon to a codon for serine and leucine bound to the tRNA.

The translation system according to the present invention may be a translation system using cells or a cell-free translation system or an in vivo translation system. Moreover, the present invention may be a kit comprising the translation system. The kit may comprise articles same as those in the translation system or may further comprise a reaction buffer, a reaction vessel, an instruction, or the like.

For example, the translation system according to the present invention may be a translation system using cultured cells derived from Escherichia coli, yeast, insect cells, or an animal. For example, in the case of Escherichia coli, a translation system can be obtained by replacing the existing tRNA genes in the genome with genes of tRNAs having anticodons to codons after the interchange and recognized by aminoacyl-tRNA synthetases corresponding to the amino acids encoded by the codons before the interchange. Aminoacyl-tRNA synthetases and amino acids to be bound to tRNA may be those that Escherichia coli originally has. To obtain a protein, Escherichia coli is transformed with an expression vector in which a template nucleic acid having a sequence modified from a nucleic acid sequence encoding a polypeptide having a predetermined amino acid sequence by interchanging codons encoding at least 2 amino acids selected from serine, leucine, and alanine is inserted.

When a translation system according to the present invention is a translation system using eukaryotic cells such as insect cells, a recombination baculovirus or a recombination adenovirus comprising a template nucleic acid having a sequence modified from a nucleic acid sequence encoding a polypeptide having a predetermined amino acid sequence by interchanging codons encoding at least 2 amino acids selected from serine, leucine, and alanine is produced according to a known method and the cells are infected with the virus. In this case, proteins encoded in the genome and tRNAs are necessary to be modified similarly according to the corresponding genetic code. The tRNAs are tRNAs having anticodons to codons after the interchange and recognized by aminoacyl-tRNA synthetases corresponding to the amino acids encoded by the codons before the interchange. A polypeptide having a predetermined amino acid sequence may be expressed by using other components such as aminoacyl-tRNA synthetases that the insect cells originally have.

When a translation system according to the present invention is a translation system using animal cells, (i) a template nucleic acid having a sequence modified from a nucleic acid sequence encoding a polypeptide having a predetermined amino acid sequence by interchanging codons encoding at least 2 amino acids selected from serine, leucine, and alanine is inserted into an expression vector and the expression vector is introduced into the animal cells by electroporation, the calcium phosphate method, lipofection, or the like. In this case, proteins encoded in the genome and tRNAs are necessary to be modified similarly according to the corresponding genetic code. The tRNAs are tRNAs having anticodons to codons after the interchange and recognized by aminoacyl-tRNA synthetases corresponding to the amino acids encoded by the codons before the interchange. A polypeptide having a predetermined amino acid sequence may be expressed by using other components such as aminoacyl-tRNA synthetases that the animal cells originally have.

When a cell-free translation system is used, for example, a known system using an Escherichia coli extract and a wheat germ extract may be used. Besides these, a rabbit erythrocyte extract and an insect cell extract may be used. In this case, a design to prevent an amino acid whose codon is exchanged from binding to the tRNAs that bind to the amino acid in the natural world. More specifically, the translation system of the first aspect is designed to prevent the aminoacyl-tRNAs in which serine is bound to tRNA^{Ser} and the aminoacyl-tRNAs in which leucine is bound to tRNA^{Leu} from being contained in the translation system and from being produced in the translation system.

The translation system according to the present invention may be a reconstituted cell-free translation system. The reconstituted cell-free translation system is a translation system constructed by reconstitution involving purified ribosomal proteins, aminoacyl-tRNA synthetases, ribosomal RNAs, amino acids, rRNAs, GTP, ATP, translation initiation factors (IF), elongation factors (EF), release factors (RF) and a ribosome recycling factor (RRF) and other factors necessary for translation. To perform transcription from DNA as well, the system may be a system containing a RNA polymerase.

When the translation system according to the present invention is a reconstituted cell-free translation system, (i) a template nucleic acid having a sequence modified from a nucleic acid sequence encoding a polypeptide having a predetermined amino acid sequence by interchanging codons encoding at least 2 amino acids selected from serine, leucine, and alanine; and (ii) aminoacyl-tRNAs having anticodons to codons after the interchange and to which the amino acids encoded by the codons before the interchange are bound are added to the translation system. Instead of (ii) aminoacyl-tRNAs having anticodons to codons after the interchange and to which the amino acids encoded by the codons before the interchange are bound, (iii) tRNAs having anticodons to codons after the interchange and recognized by aminoacyl-tRNA synthetases corresponding to the amino acids encoded by the codons before the interchange or nucleic acids encoding the tRNAs, the amino acids, and the aminoacyl-tRNA synthetases or nucleic acids encoding the aminoacyl-tRNA synthetases may be added.

Other amino acids may be added to the translation system as aminoacyl-tRNAs in which the amino acids are bound to the tRNAs to which the amino acids are bound in the natural world or the tRNAs corresponding to the amino acids, to which the amino acids are bound in the natural world, or nucleic acids encoding the tRNAs, aminoacyl-tRNA synthetases or nucleic acids encoding the aminoacyl-tRNA synthetases, and the amino acids may be added to the translation system. In the latter case, amino acids and corresponding tRNAs are bound by aminoacyl-tRNA synthetases to produce aminoacyl-tRNAs.

In the case of a reconstituted cell-free translation system, it is possible to prevent the system from containing unnecessary components depending on the purpose and therefore, for example in the translation system of the first aspect, tRNA^{Ser} and tRNA^{Leu} can be excluded from the beginning. In this way, translation into proteins in which serine and leucine are interchanged according to the interchanged codons can be prevented.

When a translation system according to the present invention is a cell-free translation system, the amino acids other than those for which codons are interchanged are translated according to the universal genetic code as usual and serine, leucine and/or alanine are encoded by interchanged codons by incubating the system under the conditions that allows the expression of protein and as a result, a predetermined protein are produced by translation as the original amino acid sequence.

The translation system using cells or cell-free translation system according to the present invention allows safe expression of proteins (for example, antibiotics resistance enzymes and toxic proteins) of which gene flow into the natural world is feared.

The translation system according to the present invention may be an in vivo translation system. For example, J. Craig Venter et al. generated a bacterium having a chemical synthesized genome. Specifically, the genome of Mycoplasma mycoides consisting of 106 base pairs is subdivided and chemically synthesized, and ligated using yeast to generate the genome (Gibson D G et al., Science Vol. 329, p. 52-56 (2010)). Interchanging at least 2 codons selected from serine, leucine, and alanine on the genome and placing genes of tRNAs in which the anticodon is modified on the genome in this method would allow the generation of a living organism (a microorganism such as microbes, a plant, or an animal) according to a new genetic code.

Living organisms having the translation system according to the present invention are safe artificial living organisms that do not exchange any genes with other living organisms on the earth. Application of such living organisms to, for example, genetically modified living organisms such as genetically modified microorganisms and genetically modified plants prevents transfer of modified genes to plants in the natural world to express harmful polypeptides and transfer of a harmful gene (for example, an infectious virus) into the genetically modified living organisms to express a harmful polypeptide.

The disclosure of all Patent Literature and Non-Patent Literature cited herein is incorporated herein by reference in their entirety.

### Examples

The present invention will be specifically described based on Examples, but the present invention is not limited to these by any means. Those skilled in the art can modify the present invention to various aspects without departing from the spirit of the present invention and such modifications are also within the scope of the present invention.

1. Outline of Design of Genetic Code and tRNA

An outline of new genetic code 1 and 2 used in Examples are illustrated in FIG. 2. In the universal genetic code (FIG. 2A), Ala is encoded by the GCU and GCC codons, Leu is encoded by the CUU and CUC codons, and Ser is encoded by the UCU and UCC codons.

In new genetic code 1 (FIG. 2B lower panel), Ala is encoded by the GCU and GCC codons as the universal genetic code, Leu is encoded by the UCU and UCC codons, and Ser is encoded by the CUU and CUC codons. To perform translation using new genetic code 1, tRNAs in which the anticodon loops of tRNA^{Ser} and tRNA^{Leu} were produced (FIG. 2B upper panel).

In new genetic code 2 (FIG. 2C lower panel), Ala is encoded by the CUU and CUC codons as the universal genetic code, Leu is encoded by the UCU and UCC codons, and Ser is encoded by the GCU and GCC codons. To perform translation using new genetic code 2, tRNAs in which the anticodon loops of tRNA^{Ala}, tRNA^{Ser}, and tRNA^{Leu} were produced (FIG. 2C upper panel). Specific methods of the experiments will be described below.

### 2. Method

### 2-1. Preparation of Reconstituted Cell-Free Translation System

Creatine kinase, creatine phosphatase, and Escherichia coli tRNAs were purchased from Roche Diagnostics. Myokinase was purchased from Sigma-Aldrich Co. LLC. Ribosomes were purified from E. coli strain A19 by using a method following the method of Clemons et al. (Clemons, W. M. et al., J. Mol. Biol., 310, 827-843, doi:10.1006/jmbi.2001.4778 (2001).). Compounds and proteins for translation were prepared according to known methods (Josephson, K., Hartman, M. C. T. & Szostak, J. W., J. Am. Chem. Soc. 127, 11727-11735, doi:10.1021/ja0515809 (2005).; Baggott, J. E., Biochem. J. 308, 1031-1036 (1995).; Shimizu, Y. et al. Nat. Biotechnol. 19, 751-755, doi:10.1038/90802 (2001).). These translation factors were stored according to the method of Ishizawa (Ishizawa, T., Kawakami, T., Reid, P. C. & Murakami, H., J. Am. Chem. Soc., 135, 5433-5440 (2013).) and used at final concentrations as set forth in the table below.

TABLE 2

| Name | Reaction mixture (μM) |
|---|---|
| AlaRS | 5.26 |
| ArgRS | 0.22 |
| AsnRS | 2.74 |
| AspRS | 0.94 |
| CysRS | 0.14 |
| GlnRS | 0.43 |
| GluRS | 1.66 |
| GlyRS | 0.65 |
| HisRS | 0.14 |
| IleRS | 2.88 |
| LeuRS | 0.29 |
| LysRS | 0.79 |
| MetRS | 0.22 |
| PheRS | 4.9 |
| ProRS | 1.15 |
| SerRS | 0.29 |
| ThrRS | 0.65 |
| TrpRS | 0.22 |
| TyrRS | 0.14 |
| ValRS | 0.14 |
| Methionyl-tRNA formyltransferases | 0.5 |
| Initiation factor 1 | 2.3 |
| Initiation factor 2 | 0.33 |
| Initiation factor 3 | 1.3 |
| Elongation factor G | 0.22 |
| Elongation factor Tu | 8.3 |
| Elongation factor Ts | 8.3 |
| Release factor 2 | 0.21 |
| Release factor 3 | 0.14 |
| Ribosome recycling factor | 0.42 |
| Nucleoside-diphosphate kinase | 0.083 |
| Inorganic pyrophosphatase | 0.083 |
| T7 RNA polymerase | 0.083 |
| Creatine kinase | 3.3 (μg/mL) |
| Myokinase | 2.5 (μg/mL) |
| Ribosome | 1 |
| ATP | 2 |
| GTP | 2 |
| CTP | 1 |
| UTP | 1 |
| Creatine phosphate | 20.1 |
| Hepes-KOH pH 7.6 | 50.5 |
| Potassium acetate | 100.9 |
| Magnesium acetate | 15.1 |
| Spermidine | 2 |
| DTT | 1 |
| 10-formyl-5,6,7,8 tetrahydrofolic acid | 0.1 |

### 2-2. Expression and Purification of C5 Protein

Escherichia coli (E. coli Rosetta 2) was transformed with the plasmid pET21a containing the gene of C5 protein by a conventional method. The transformed Escherichia coli was suspended on an agar plate containing 100 μg/mL ampicillin, 20 μg/mL chloramphenicol and 1% (w/v) glucose and incubated at 37° C. overnight. The culture of Escherichia coli was grown until an OD₅₉₀ of over approximately 2.0 in LB medium (40 mL) containing 100 μg/mL ampicillin, 20 μg/mL chloramphenicol, and 1% (w/v) glucose at 37° C. 20 mL of the Escherichia coli culture was added to LB medium (500 mL) containing 100 μg/mL ampicillin, 20 μg/mL chloramphenicol, and 1% (w/v) glucose and grown until an OD₅₉₀ of 0.79 at 37° C. 0.5 mM IPTG was added to induce the expression of C5 protein and incubated at 25° C. overnight. Cells were collected by centrifugation at 9000 rpm at 4° C. for 2 minutes, and resuspended in 70 mL of B1 buffer (300 mM KCl, 50 mM Tris-HCl pH 8.0, 5 mM imidazole pH 7.5, 0.2 mM DTT, 10 mM MgCl₂, 1 M NH₄Cl, 0.25% (v/v) Tween 20) containing 12 mg of phenylmethylsulfonyl fluoride dissolved in 600 μL of ethanol. Cells were disrupted by 6-minute sonication and disrupted cells were centrifuged at 4° C. at 13,000 rpm, for 15 minutes. The supernatant was collected, 21 g of (NH₄)₂SO₄ was added thereto, and the mixture was centrifuged at 4° C. at 13,000 rpm, for 5 minutes. The precipitate was dissolved in 10 mL of B1 buffer. The C5 protein was purified with a Ni-affinity column and eluted with the elution buffer (300 mM KCl, 20 mM Hepes-K pH 7.5, 250 mM imidazole pH 7.5, 0.2 mM DTT, 10 mM MgCl₂, 1 M NH₄Cl, 0.25% (v/v) Tween 20). The concentration of C5 protein was measured by the absorbance at 280 nm and then the C5 protein was stored in 20% glycerol at −80° C.

2-3. Preparation of tRNA

The elongation (10 μL) was performed under the following conditions: 1×PCR buffer (10 mM Tris-HCl pH 8.4, 100 mM KCl, 0.1% (v/v) Triton X-100, 2 mM MgSO₄, 0.2 mM each of dNTPs (dATP, dTTP, dGTP, dCTP)), 1 μM each of eX.F primers, 1 μM each of eX.R primers, 1.6 nM Phusion DNA polymerase (see Table below).

TABLE 3

| tRNA number | ex. F | ex. R | PCR1.R | PCR2.R |
|---|---|---|---|---|
| 0 | fMet 1G (CAT)-5.F50 | fMet 1G (CAT)at.R43 | fMet 1G (CAT)-3.R38 | fMet 1G (CAT)-3.R20 |
| 1 | Ala2 (GGC)-5.F49 | Ala2 (GGC)at.R43 | Ala2 (GGC)-3.R38 | Ala2 (GGC)-3.R20 |
| 2 | Arg2 (ACG−>GCG)-5.F50 | Arg2 (ACG−>GCG)at.R43 | Arg2 (ACG−>GCG)-3.R38 | Arg2 (ACG−>GCG)-3.R20 |
| 3 | Asn 1G72C (GTT)-5.F49 | Asn 1G72C (GTT)at.R43 | Asn 1G72C (GTT)-3.R38 | Asn 1G72C (GTT)-3.R20 |
| 4 | Asp (GTC)-5.F50 | Asp (GTC)at.R43 | Asp (GTC)-3.R38 | Asp (GTC)-3.R20 |
| 5 | Cys (GCA)-5.F48 | Cys (GCA)at.R43 | Cys (GCA)-3.R37 | Cys (GCA)-3.R20 |
| 6 | Gln2 (CTG)-5\|.F49 | Gln2 (CTG)at.R43 | Gln2 (CTG)-3.R38 | Gln2 (CTG)-3.R20 |
| 7 | Glu (TTC−>CTC)-5.F50 | Glu (TTC−>CTC)at.R43 | Glu (TTC−>CTC)-3.R37 | Glu (TTC−>CTC)-3.R20 |
| 8 | Gly3 (GCC)-5.F49 | Gly3 (GCC)at.R43 | Gly3 (GCC)-3.R38 | Gly3 (GCC)-3.R20 |
| 9 | His (GTG)-5.F50 | His (GTG)at.R43 | His (GTG)-3.R38 | His (GTG)-3.R20 |
| 10 | Ile (GAT) 1G72C-5.F50 | Ile 1G72C (GAT)at.R43 | Ile 1G72C(GAT)-3.R38 | Ile 1G72C(GAT)-3.R20 |
| 11 | Leu2 (GAG)-5.F50 | Leu2 (GAG)at.R43 | Leu2 (GAG)-3.R48 | Leu2 (GAG)-3.R20 |
| 12 | Lys (TTT−>CTT)-5.F49 | Lys (TTT−>CTT)at.R43 | Lys (TTT−>CTT)-3.R38 | Lys (TTT−>CTT)-3.R20 |
| 13 | Met (CAT)-5.F50 | Met (CAT)at.R43 | Met (CAT)-3.R38 | Met (CAT)-3.R20 |
| 14 | Phe 32C (GAA)-5.F49 | Phe 32C (GAA)at.R43 | Phe 32C (GAA)-3.R38 | Phe 32C (GAA)-3.R20 |
| 15 | Pro2 (GGG)-5\|.F50 | Pro2 (GGG)at.R43 | Pro2 (GGG)-3.R38 | Pro2 (GGG)-3.R20 |
| 16 | Ser5 (GGA)-5.F50 | Ser5 (GGA)at.R43 | Ser5 (GGA)-3.R49 | Ser5 (GGA)-3.R20 |
| 17 | Thr3 (GGT)-5.F49 | Thr3 (GGT)at.R43 | Thr3 (GGT)-3.R38 | Thr3 (GGT)-3.R20 |
| 18 | Trp (CCA)-5\|.F49 | Trp (CCA)at.R43 | Trp (CCA)-3.R38 | Trp (CCA)-3.R20 |
| 19 | Tyr (GTA)-5.F50 | Tyr (GTA)at.R43 | Tyr (GTA)-3.R46 | Tyr (GTA)-3.R20 |
| 20 | Val2 (GAC)-5.F50 | Val2 (GAC)at.R43 | Val2 (GAC)-3.R38 | Val2 (GAC)-3.R20 |
| 21 | Leu2 (GAG)-5.F50 | nLeu2S (GAG−>GGA)at.R43 | Leu2 (GAG)-3.R48 | Leu2 (GAG)-3.R20 |
| 22 | Ser2 (CGA)-5.F50 | nSer2L (CGA−>GAG)at.R43 | Ser2 (CGA)-3.R51 | Ser2 (CGA)-3.R20 |
| 23 | Ala2 (GGC)-5.F49 | nAla2L (GGC−>GAG)at.R43 | Ala2 (GGC)-3.R38 | Ala2 (GGC)-3.R20 |
| 24 | Ser2 (CGA)-5.F50 | nSer2A (CGA−>GGC)at.R43 | Ser2 (CGA)-3.R51 | Ser2 (CGA)-3.R20 |

TABLE 4

| Cate- | | |
|---|---|---|
| gory | Primer name | Sequence |
| ex.F | fMet 1G | 5′-GTAAT ACGAC |
| | (CAT)-5.F50 | TCACT ATAGG CGGGG |
| | | TGGAG CAGCC TGGTA |
| | | GCTCG TCGGG-3′ |
| | Ala2 | 5′-GTAAT ACGAC |
| | (GGC)-5.F49 | TCACT ATAGG GGCTA |
| | | TAGCT CAGCT GGGAG |
| | | AGCGC TTGC-3′ |
| | Arg2 | 5′-GTAAT ACGAC |
| | (ACG->GCG)-5.F50 | TCACT ATAGC ATCCG |
| | | TAGCT CAGCT GGATA |
| | | GAGTA CTCGG-3′ |
| | Asn 1G72C | 5′-GTAAT ACGAC |
| | (GTT)-5.F49 | TCACT ATAGC CTCTG |
| | | TAGTT CAGTC GGTAG |
| | | AACGG CGGA-3′ |
| | Asp | 5′-GTAAT ACGAC |
| | (GTC)-5.F50 | TCACT ATAGG AGCGG |
| | | TAGTT CAGTC GGTTA |
| | | GAATA CCTGC-3′ |
| | Cys | 5′-GTAAT ACGAC |
| | (GCA)-5.F48 | TCACT ATAGG CGCGT |
| | | TAACA AAGCG GTTAT |
| | | GTAGC GGA-3′ |
| | Gln2 | 5′-GGAAC GCGCG |
| | (CTG)-5I.F49 | ACTCT AATTG GGGTA |
| | | TCGCC AAGCG GTAAG |
| | | GCACC GGA-3′ |
| | Glu | 5′-GTAAT ACGAC |
| | (TTC->CTC)-5.F50 | TCACT ATAGT CCCCT |
| | | TCGTC TAGAG GCCCA |
| | | GGACA CCGCC-3′ |
| | Gly3 | 5′-GTAAT ACGAC |
| | (GCC)-5.F49 | TCACT ATAGC GGGAA |
| | | TAGCT CAGTT GGTAG |
| | | AGCAC GACC-3′ |
| | His | 5′-GTAAT ACGAC |
| | (GTG)-5.F50 | TCACT ATAGG TGGCT |
| | | ATAGC TCAGT TGGTA |
| | | GAGCC CTGGA-3′ |
| | Ile (GAT) | 5′-GTAAT ACGAC |
| | 1G72C.5.F50 | TCACT ATAGG GCTTG |
| | | TAGCT CAGGT GGTTA |
| | | GAGCG CACCC-3′ |
| | Leu2 | 5′-GTAAT ACGAC |
| | (GAG)-5.F50 | TCACT ATAGC CGAGG |
| | | TGGTG GAATT GGTAG |
| | | ACACG CTACC-3′ |
| | Lys | 5′-GTAAT ACGAC |
| | (TTT->CTT)-5.F49 | TCACT ATAGG GTCGT |
| | | TAGCT CAGTT GGTAG |
| | | AGCAG TTGA-3′ |
| | Met | 5′-GTAAT ACGAC |
| | (CAT)-5.F50 | TCACT ATAGG CTACG |
| | | TAGCT CAGTT GGTTA |
| | | GAGCA CATCA-3′ |
| | Phe | 5′-GTAAT ACGAC |
| | 32C (GAA)-5.F49 | TCACT ATAGC CCGGA |
| | | TAGCT CAGTC GGTAG |
| | | AGCAG GGGA-3′ |
| | Pro.2 | 5′-GGAAC GCGCG |
| | (GGG)-5I.F50 | ACTCT AATCG GCACG |
| | | TAGCG CAGCC TGGTA |
| | | GCGCA CCGTC-3′ |
| | Ser5 | 5′-GTAAT ACGAC |
| | (GGA)-5.F50 | TCACT ATAGG TGAGG |
| | | TGTCC GAGTG GCTGA |
| | | AGGAG CACGC-3′ |
| | Thr3 | 5′-GTAAT ACGAC |
| | (GGT)-5.F49 | TCACT ATAGC TGATA |
| | | TAGCT CAGTT GGTAG |
| | | AGCGC ACCC-3′ |
| | Trp | 5′-GGAAC GCGCG |
| | (CCA)-5I.F49 | ACTCT AATAG GGGCG |
| | | TAGTT CAATT GGTAG |
| | | AGCAC CGGT-3′ |
| | Tyr | 5′-GTAAT ACGAC |
| | (GTA)-5.F50 | TCACT ATAGG TGGGG |
| | | TTCCC GAGCG GCCAA |
| | | AGGGA GCAGA-3′ |
| | Val2 | 5′-GTAAT ACGAC |
| | (GAC)-5.F50 | TCACT ATAGC GTCCG |
| | | TAGCT CAGTT GGTTA |
| | | GAGCA CCACC-3′ |
| ex.R | Met 1G | 5′-GAACC GACGA |
| | (CAT)at.R43 | TCTTC GGGTT ATGAG |
| | | CCCGA CGAGC TACCA |
| | | GGC-3′ |
| | Ala2 | 5′-GAACC GCTGA |
| | (GGC)at.R43 | CCTCT TGCAT GCCAT |
| | | GCAAG CGCTC TCCCA |
| | | GCT-3′ |
| | Arg2 | 5′-GAACC TCCGA |
| | (ACG->GCG)at.R43 | CCGCT CGGTT CGCAG |
| | | CCGAG TACTC TATCC |
| | | AGC-3′ |
| | Asn 1G72C | 5′-GAACC AGTGA |
| | (GTT)at.R43 | CATAC GGATT AACAG |
| | | TCCGC CGTTC TACCG |
| | | ACT-3′ |
| | Asp | 5′-GAACC CGCGA |
| | (GTC)at.R43 | CCCCC TGCGT GACAG |
| | | GCAGG TATTC TAACC |
| | | GAC-3′ |
| | Cys | 5′-CGAAC CGGAC |
| | (GCA)at.R43 | TAGAC GGATT TGCAA |
| | | TCCGC TACAT AACCG |
| | | CTT-3′ |
| | Gln2 | 5′-GAACC TCGGA |
| | (CTG)at.R43 | ATGCC GGAAT CAGAA |
| | | TCCGG TGCCT TACCG |
| | | CTT-3′ |
| | Glu | 5′-CGAAC CCCTG |
| | (TTC)->CTC)at.R43 | TTACC GCCGT GAGAG |
| | | GGCGG TGTCC TGGGC |
| | | CTC-3′ |
| | Gly3 | 5′-GAACT CGCGA |
| | (GCC)at.R43 | CCCCG ACCTT GGCAA |
| | | GGTCG TGCTC TACCA |
| | | ACT-3′ |
| | His | 5′-GAACC CACGA |
| | (GTG)at.R43 | CAACT GGAAT CACAA |
| | | TCCAG GGCTC TACCA |
| | | ACT-3′ |
| | Ile 1G72C | 5′-GAACC ACCGA |
| | (GAT)at.R43 | CCTCA CCCTT ATCAG |
| | | GGGTG CGCTC TAACC |
| | | ACC-3′ |
| | Leu2 | 5′-GCCCT ATTGG |
| | (GAG)at.R43 | GCACT ACCAC CTCAA |
| | | GGTAG CGTGT CTACC |
| | | AAT-3′ |
| | Lys | 5′-GAACC TGCGA |
| | (TTT->CTT)at.R43 | CCAAT TGATT AAGAG |
| | | TCAAC TGCTC TACCA |
| | | ACT-3′ |
| | Met | 5′-GAACC TGTGA |
| | (CAT)at.R43 | CCCCA TCATT ATGAG |
| | | TGATG TGCTC TAACC |
| | | AAC-3′ |
| | Phe 32C | 5′-GAACC AAGGA |
| | (GAA)at.R43 | CACGG GGATT TTCAG |
| | | TCCCC TGCTC TACCG |
| | | ACT-3′ |
| | Pro2 | 5′-GAACC TCCGA |
| | (GGG)at.R43 | CCCCC GACAC CCCAT |
| | | GACGG TGCGC TACCA |
| | | GGC-3′ |
| | Ser5 | 5′-ACGTT GCCGT |
| | (GGA)at.R43 | ATACA CACTT TCCAG |
| | | GCGTG CTCCT TCAGC |
| | | CAC-3′ |
| | Thr3 | 5′-GAACT GCCGA |
| | (GGT)at.R43 | CCTCA CCCTT ACCAA |
| | | GGGTG CGCTC TACCA |
| | | ACT-3′ |
| | Trp | 5′-GAACT CCCAA |
| | (CCA)at.R43 | CACCC GGTTT TGGAG |
| | | ACCGG TGCTC TACCA |
| | | ATT-3′ |
| | Tyr | 5′-GAAGT CTGTG |
| | (GTA)at.R43 | ACGGC AGATT TACAG |
| | | TCTGC TCCCT TTGGC |
| | | CGC-3′ |
| | Val2 | 5′-GAACC ACCGA |
| | (GAC)at.R43 | CCCCC ACCAT GTCAA |
| | | GGTGG TGCTC TAACC |
| | | AAC-3′ |
| | nLeu2S | 5′-GCCCT ATTGG |
| | (GAG->GGA)at.R43 | GCACT ACCTT TCGAG |
| | | GGTAG CGTGT CTACC |
| | | AAT-3′ |
| | nSer2L | 5′-AGTTG CCCCT |
| | (CGA->GAG)at.R43 | ACTCC GGTAC CTCAA |
| | | ACCGG TCCGT TCAGC |
| | | CGC-3′ |
| | nAla2L | 5′-GAACC GCTGA |
| | (GGC->GAG)at.R43 | CCTCT TGCAC CTCAA |
| | | GCAAG CGCTC TCCCA |
| | | GCT-3′ |
| | nSer2A | 5′-AGTTG CCCCT |
| | (CGA->GGC)at.R43 | ACTCC GGTAT GCCAT |
| | | ACCGG TCCGT TCAGC |
| | | CGC-3′ |
| PCR1.R | 1Met 1G | 5′-TGGTT GCGGG |
| | (CAT)-3.R38 | GGCCG GATTT GAACC |
| | | GACGA TCTTC GGG-3′ |
| | Ala2 | 5′-TGGTG GAGCT |
| | (GGC)-3.R38 | AAGCG GGATC GAACC |
| | | GCTGA CCTCT TGC-3′ |
| | Arg2 | 5′-TGGTG CATCC |
| | (ACG->GCG)-3.R38 | GGGAG GATTC GAACC |
| | | TCCGA CCGCT CGG-3′ |
| | Asn 1G72C | 5′-TGGCG CCTCT |
| | (GTT)-3.R38 | GACTG GACTC GAACC |
| | | AGTGA CATAC GGA-3′ |
| | Asp | 5′-TGGCG GAACG |
| | (GTC)-3.R38 | GACGG GACTC GAACC |
| | | CGCGA CCCCC TGC-3′ |
| | Cys | 5′-TGGAG GCGCG |
| | (GCA)-3.R38 | TTCCG GAGTC GAACC |
| | | GGACT AGACG GA-3′ |
| | Gln2 | 5′-TGGCT GGGGT |
| | (CTG)-3.R38 | ACGAG GATTC GAACC |
| | | TCGGA ATGCC GGA-3′ |
| | Glu | 5′-TGGCG TCCCC |
| | (TTC->CTC-3.R37 | TAGGG GATTC GAACC |
| | | CCTGT TACCG CC-3′ |
| | Gly3 | 5′-TGGAG CGGGA |
| | (GCC)-3.R38 | AACGA GACTC CAACT |
| | | CGCGA CCCCG ACC-3′ |
| | His | 5′-TGGGG TGGCT |
| | (GTG)-3.R38 | AATGG GATTC GAACC |
| | | CACGA CAACT GGA-3′ |
| | Ile 1G72C | 5′-TGGTG GGCCT |
| | (GAT)-3.R38 | GAGTG GACTT GAACC |
| | | ACCGA CCTCA CCC-3′ |
| | Leu2 | 5′-TGGTA CCGAG |
| | (GAG)-3.R48 | GACGG GACTT GAACC |
| | | CGTAA GCCCT ATTGG |
| | | GCACT ACC-3′ |
| | Lys | 5′-TGGTG GGTCG |
| | (TTT->CTT)-3.R38 | TGCAG GATTC GAACC |
| | | TGCGA CCAAT TGA-3′ |
| | Met | 5′-TGGTG GCTAC |
| | (CAT)-3.R38 | GACGG GATTC GAACC |
| | | TGTGA CCCCA TCA-3′ |
| | Phe 32C | 5′-TGGTG CCCGG |
| | (GAA-3.R38 | ACTCG GAATC GAACC |
| | | AAGGA CACGG GGA-3′ |
| | Pro2 | 5′-TGGTC GGCAC |
| | (GGG)-3.R38 | GAGAG GATTT GAACC |
| | | TCCGA CCCCC GAC-3′ |
| | Ser2 | 5′-TGGCG GAGAG |
| | (CGA)-3.R51 | AGGGG GATTT GAACC |
| | | CCCGG TAGAG TTGCC |
| | | CCTAC TCCGG T-3′ |
| | Ser5 | 5′-TGGCG GTGAG |
| | (GGA)-3.R49 | GGGGG GATTC GAACC |
| | | CCCGA TACGT TGCCG |
| | | TATAC ACAC-3′ |
| | Thr3 | 5′-TGGTG CTGAT |
| | (GGT)-3.R38 | AGGCA GATTC GAACT |
| | | GCCGA CCTCA CCC-3′ |
| | Trp | 5′-TGGCA GGGGC |
| | (CCA)-3.R38 | GGAGA GACTC GAACT |
| | | CCCAA CACCC GGT-3′ |
| | Tyr | 5′-TGGTG GTGGG |
| | (GTA)-3.R46 | GGAAG GATTC GAACC |
| | | TTCGA AGTCT GTGAC |
| | | GGCAG A-3′ |
| | Val2 | 5′-TGGTG CGTCC |
| | (GAC)-3.R38 | GAGTG GACTC GAACC |
| | | ACCGA CCCCC ACC-3′ |
| PCR2.R | 1Met 1G | 5′-TGGTT GCGGG |
| | (CAT)-3.R20 | GGCCG GATTT-3′ |
| | Ala | 5′-TGGTG GAGCT |
| | (GGC)-3.R20 | AAGCG GGATC-3′ |
| | Arg2 | 5′-TGGTG CATCC |
| | (ACG->GCG)-3.R20 | GGGAG GATTC-3′ |
| | Asn 1G72C | 5′-TGGCG CCTCT |
| | (GTT)-3.R20 | GACTG GACTC-3′ |
| | Asp | 5′-TGGCG GAACG |
| | (GTC)-3.R20 | GACGG GACTC-3′ |
| | Cys | 5′-TGGAG GCGCG |
| | (GCA)-3.R20 | TTCCG GAGTC-3′ |
| | Gln2 | 5′-TGGCT GGGGT |
| | (GTC)-3.R20 | ACGAG GATTC-3′ |
| | Glu | 5′-TGGCG TCCCC |
| | (TTC->CTC)-3.R20 | TAGGG GATTC-3′ |
| | Gly3 | 5′-TGGAG CGGGA |
| | (GCC)-3.R20 | AACGA GACTC-3′ |
| | His | 5′-TGGGG TGGCT |
| | (GTG)-3.R20 | AATGG GATTC-3′ |
| | Ile 1G72C | 5′-TGGTG GGCCT |
| | (GAT)-3.R20 | GAGTG GACTT-3′ |
| | Leu2 | 5′-TGGTA CCGAG |
| | (GAG)-3.R20 | GACGG GACTT-3′ |
| | Lys | 5′-TGGTG GGTCG |
| | (TTT->CTT)-3.R20 | TGCAG GATTC-3′ |
| | Met | 5′-TGGTG GCTAC |
| | (CAT)-3.R20 | GACGG GATTC-3′ |
| | Phe 32C | 5′-TGGTG CCCGG |
| | (GAA)-3.R20 | ACTCG GAATC-3′ |
| | Pro2 | 5′-TGGTC GGCAC |
| | (GGG)-3.R20 | GAGAG GATTT-3′ |
| | Ser2 | 5′-TGGCG GAGAG |
| | (CGA)-3.R20 | AGGGG GATTT-3′ |
| | Ser5 | 5′-TGGCG GTGAG |
| | (GGA)-3.R20 | GGGGG GATTC-3′ |
| | Thr3 | 5′-TGGTG CTGAT |
| | (GGT)-3.R20 | AGGCA GATTC-3′ |
| | Trp | 5′-TGGCA GGGGC |
| | (CCA)-3.R20 | GGAGA GACTC-3′ |
| | Tyr | 5′-TGGTG GTGGG |
| | (GTA)-3.R20 | GGAAG GATTC-3′ |
| | Val2 | 5′-TGGTG CGTCC |
| | (GAC)-3.R20 | GAGTG GACTC-3′ |

The aforementioned solution was heated at 94° C. for 3 minutes and then elongation was performed by repeating 5 cycles of 50° C. for 1 minute and 72° C. for 2 minutes.

### 2-4. Amplification

To prepare pre-tDNA^{Gln}, pre-tDNA^{Pro}, and pre-tDNA^{Trp}, the first PCR (800 μL) was performed under the following conditions: 1×PCR buffer, 0.5 μM T7pro-leader.F36, 0.5 μM each of PCR1.R primers, 1.6 nM Phusion DNA polymerase, 2.5% (v/v) of the extension mixture (see Table above). To prepare other tDNAs, the first PCR (200 μL) was performed under the following conditions: 1×PCR buffer, 0.5 μM T7ex5.F22, 0.5 μM each of PCR1.R primers, 1.6 nM Phusion DNA polymerase, 2.5% (v/v) of the extension mixture.

The aforementioned solution was heated at 94° C. for 3 minutes and then DNA was amplified by repeating 12 cycles of 94° C. for 20 seconds, 50° C. for 20 seconds, and 72° C. for 30 seconds.

The second PCR (3.2 mL of tDNA^{Lys}, 2.4 mL of pre-tDNA^{Gln}, pre-tDNA^{Pro}, pre-tDNA^{Trp}, 1.6 mL of other tDNAs) was performed under the following conditions: 1×PCR buffer, 1 μM T7ex5.F22, 1 μM each of PCR2.R primers, 1.6 nM Phusion DNA polymerase, 0.5% (v/v) of the first PCR solution (see Tables 3 and 4).

This solution was heated at 94° C. for 3 minutes and then DNA was amplified by repeating 12 cycles of 94° C. for 20 seconds, 50° C. for 20 seconds, and 72° C. for 30 seconds.

### 2-5. In Vitro Transcription and Purification

In vitro transcription (tRNA^{Lys}: 24 mL, pre-tRNA^{Gln}: 12 mL, pre-tRNA^{Pro}, pre-tRNA^{Trp} and other tRNAs: 8 mL) was performed under the following conditions: 1×T7 buffer (40 mM Tris-HCl pH 8.0, 1 mM spermidine, 5 mM KCl, 0.01% (v/v) Triton X-100, 10 mM DTT), 22.5 mM MgCl₂, 3.25 mM each of NTPs (ATP, TTP, GTP, CTP), 5 mM GMP, 0.3 μM T7 RNA polymerase, about 20% (v/v) of the second PCR solution. The reaction solution was incubated at 37° C. for 12 hours. pre-tRNA^{Gln} pre-tRNA^{Pro}, and pre-tRNA^{Trp} were cleaved with RNase P (see below). Other tRNAs were mixed with 16 μL of DNase I (10 u/μL) and 16 μL of 3 M MgCl₂) and the mixtures were incubated at 37° C. for 36 hours. After the phenol/chloroform extraction, 400 μL of 0.5 M EDTA pH 8.0 and 800 μL of 3 M NaCl were added to the solution. 8 mL of 2-propanol was added to precipitate the tRNA. The precipitate was dissolved in 10 mL of 0.3 M NaCl and 8 mL of 2-propanol was added to precipitate the tRNA. After washing with 70% ethanol, the tRNA was dissolved in 500 μL of ultrapure water. The concentration of each tRNA was measured by the absorbance at 260 nm. All sequences of the in vitro transcribed tRNAs are set forth in Table below.

TABLE 5

| tRNA | | | | | | |
|---|---|---|---|---|---|---|
| num- | Amino | | Length | | Anti- | |
| ber | acid | Sc | (mer) | Codon | codon | Sequence |
| 0 | fMet | 82.92 | 77 | AUG | CAU | 5′-GGCGG GGUGG |
| | | | | | | AGCAG CCUGG UAGCU |
| | | | | | | CGUCG GGCUC AUAAC |
| | | | | | | CCGAA GAUCG UCGGU |
| | | | | | | UCAAA UCCGG CCCCC |
| | | | | | | GCAAC CA-3′ |
| 1 | Ala | 86.51 | 76 | GCU, | GGC | 5′-GGGGC UAUAG |
| | | | | GCC | | CUCAG CUGGG AGAGC |
| | | | | | | GCUUG CAUGG CAUGC |
| | | | | | | AAGAG GUCAG GGGUU |
| | | | | | | CGAUC CCGCU UAGCU |
| | | | | | | CCACC A-3′ |
| 2 | Arg | 87.22 | 77 | CGU, | GCG | 5′-GCAUC CGUAG |
| | | | | CGC | | CUCAG CUGGA UAGAG |
| | | | | | | UACUC GGCUG CGAAC |
| | | | | | | CGAGC GGUCG GAGGU |
| | | | | | | UCGAA UCCUC CCGGA |
| | | | | | | UGCAC CA-3′ |
| 3 | Asn | 87.13 | 76 | AAU, | GUU | 5′-GCCUC UGUAG |
| | | | | AAC | | UUCAG UCGGU AGAAC |
| | | | | | | GGCGG ACUGU UAAUC |
| | | | | | | CGUAU GUCAC UGGUU |
| | | | | | | CGAGU CCAGU CAGAG |
| | | | | | | GCGCC A-3′ |
| 4 | Asp | 90.74 | 77 | GAU, | GUC | 5′-GGAGC GGUAG |
| | | | | GAC | | UUCAG UCGGU UAGAA |
| | | | | | | UACCU GCCUG UCACG |
| | | | | | | CAGGG GGUCG CGGGU |
| | | | | | | UGGAG UCCCG UCCGU |
| | | | | | | UCCGC CA-3′ |
| 5 | Cys | 51.48 | 74 | UGU, | GCA | 5′-GGCGC GUUAA |
| | | | | UGC | | CAAAG CGGUU AUGUA |
| | | | | | | GCGGA UUGCA AAUCC |
| | | | | | | GUCUA GUCCG GUUCG |
| | | | | | | ACUCC GGAAC GCGCC |
| | | | | | | UCCA-3′ |
| 6 | Gln | 75.83 | 75 | CAG | CUG | 5′-UGGGG UAUCG |
| | | | | | | CCAAG CGGUA AGGCA |
| | | | | | | CCGGA UUCUG AUUCC |
| | | | | | | GGCAU UCCGA GGUUC |
| | | | | | | GAAUC CUCGU ACCCC |
| | | | | | | AGCCA-3′ |
| 7 | Glu | 59.60 | 76 | GAG | CUC | 5′-GUCCC CUUCG |
| | | | | | | UCUAG AGGCC CAGGA |
| | | | | | | CACCG CCCUC UCACG |
| | | | | | | GCGGU AACAG GGGUU |
| | | | | | | CGAAU CCCCU AGGGG |
| | | | | | | ACGCC A-3′ |
| 8 | Gly | 93.74 | 76 | GGU, | GCC | 5′-GCGGG AAUAG |
| | | | | GGC | | CUCAG UUGGU AGAGC |
| | | | | | | ACGAC CUUGC CAAGG |
| | | | | | | UCGGG GUCGC GAGUU |
| | | | | | | CGAGU CUCGU UUCCC |
| | | | | | | GCUCC A-3′ |
| 9 | His | 84.86 | 77 | CAU, | GUG | 5′-GGUGG CUAUA |
| | | | | CAC | | GCUCA GUUGG UAGAG |
| | | | | | | CCCUG GAUUG UGAUU |
| | | | | | | CGAGU UGUCG UGGGU |
| | | | | | | UCGAA UCCCA UUAGC |
| | | | | | | CACCC CA-3′ |
| 10 | Ile | 88.37 | 77 | AUU, | GAU | 5′-GGGCU UGUAG |
| | | | | AUC | | CUCAG GUGGU UAGAG |
| | | | | | | CGCAC CCCUG AUAAG |
| | | | | | | GGUGA GGUCG GUGGU |
| | | | | | | UCAAG UCCAC UCAGG |
| | | | | | | CCCAC CA-3′ |
| 11 | Leu | 70.30 | 87 | CUU, | GAG | 5′-GCCGA GGUGG |
| | | | | CUC | | UGGAA UUGGU AGACA |
| | | | | | | CGCUA CCUUG AGGUG |
| | | | | | | GUAGU GCCCA AUAGG |
| | | | | | | GCUUA CGGGU UCAAG |
| | | | | | | UCCCG UCCUC GGUAC |
| | | | | | | CA-3′ |
| 12 | Lys | 99.54 | 76 | AAG | CUU | 5′-GGGUC GUUAG |
| | | | | | | CUCAG UUGGU AGAGC |
| | | | | | | AGUUG ACUCU UAAUC |
| | | | | | | AAUUG GUCGC AGGUU |
| | | | | | | CGAAU CCUGC ACGAC |
| | | | | | | GCACC A-3′ |
| 13 | Met | 96.18 | 76 | AUG | CAU | 5′-GGCUA CGUAG |
| | | | | | | CUCAG UUGGU UAGAG |
| | | | | | | CACAU CACUC AUAAU |
| | | | | | | GAUGG GGUCA CAGGU |
| | | | | | | UCGAA UCCCG UCGUA |
| | | | | | | GCCAC CA-3′ |
| 14 | Phe | 84.11 | 76 | UUU, | GAA | 5′-GCCCG GAUAG |
| | | | | UUC | | CUCAG UCGGU AGAGC |
| | | | | | | AGGGG ACUGA AAAUC |
| | | | | | | CCCGU GUCCU UGGUU |
| | | | | | | CGAUU CCGAG UCCGG |
| | | | | | | GCACC A-3′ |
| 15 | Pro | 77.97 | 77 | CCU, | GGG | 5′-CGGCA CGUAG |
| | | | | CCC | | CGCAG CCUGG UAGCG |
| | | | | | | CACCG UCAUG GGGUG |
| | | | | | | UCGGG GGUGC GAGGU |
| | | | | | | UCAAA UCCUC UCGUG |
| | | | | | | CCGAC CA-3′ |
| 16 | Ser | 68.60 | 88 | UCU, | GGA | 5′-GGUGA GGUGU |
| | | | | UCC | | CCGAG UGGCU GAAGG |
| | | | | | | AGCAC GCCUG GAAAG |
| | | | | | | UGUGU AUACG GCAAC |
| | | | | | | GUAUC GGGGG UUCGA |
| | | | | | | AUCCC CCCCU CACCG |
| | | | | | | GCA-3′ |
| 17 | Thr | 94.75 | 76 | ACU, | GGU | 5′-GCUGA UAUAG |
| | | | | ACC | | CUCAG UUGGU AGAGC |
| | | | | | | GCACC CUUGG UAAGG |
| | | | | | | GUGAG GUCGG CAGUU |
| | | | | | | CGAAU CUGCC UAUCA |
| | | | | | | GCACC A-3′ |
| 18 | Trp | 82.06 | 76 | UGG | CCA | 5′-AGGGG CGUAG |
| | | | | | | UUCAA UUGGU AGAGC |
| | | | | | | ACCGG UCUCC AAAAC |
| | | | | | | CGGGU GUUGG GAGUU |
| | | | | | | CGAGU CUCUC CGCCC |
| | | | | | | CUGCC A-3′ |
| 19 | Tyr | 67.63 | 85 | UAU, | GUA | 5′-GGUGG GGUUC |
| | | | | UAC | | CCGAG CGGCC AAAGG |
| | | | | | | GAGCA GACUG UAAAU |
| | | | | | | CUGCC GUCAC AGACU |
| | | | | | | UCGAA GGUUC GAAUC |
| | | | | | | CUUCC CCCAC |
| | | | | | | CACCA-3′ |
| 20 | Val | 96.76 | 77 | GUU, | GAC | 5′-GCGUC CGUAG |
| | | | | GUC | | CUCAG UUGGU UAGAG |
| | | | | | | CACCA CCUUG ACAUG |
| | | | | | | GUGGG GGUCG GUGGU |
| | | | | | | UCGAG UCCAC UCGGA |
| | | | | | | CGCAC CA-3′ |
| 21 | Leu | 70.30 | 87 | UCU, | GGA | 5′-GCCGA GGUGG |
| | | | | UCC | | UGGAA UUGGU AGACA |
| | | | | | | CGCUA CCCUG GAAAG |
| | | | | | | GUAGU GCCCA AUAGG |
| | | | | | | GCUUA CGGGU UCAAG |
| | | | | | | UCCCG UCCUC GGUAC |
| | | | | | | CA-3′ |
| 22 | Ser | 75.49 | 90 | CUU, | GAG | 5′-GGAGA GAUGC |
| | | | | CUC | | CGGAG CGGCU GAACG |
| | | | | | | GACCG GUUUG AGGUA |
| | | | | | | CCGGA GUAGG GGCAA |
| | | | | | | CUCUA CCGGG GGUUC |
| | | | | | | AAAUC CCCCU CUCUC |
| | | | | | | CGCCA-3′ |
| 23 | Ala | 86.51 | 76 | CUU, | GAG | 5′-GGGGC UAUAG |
| | | | | CUC | | CUCAG CUGGG AGAGC |
| | | | | | | GCUUG CUUGA GGUGC |
| | | | | | | AAGAG GUCAG CGGUU |
| | | | | | | CGAUC CCGCU UACCU |
| | | | | | | CCACC A-3′ |
| 24 | Ser | 75.49 | 90 | GCU, | GGC | 5′-GGAGA GAUGC |
| | | | | GCC | | CGGAG CGGCU GAACG |
| | | | | | | GACCG GUAUG GCAUA |
| | | | | | | CCGGA GUAGG GGCAA |
| | | | | | | CUCUA CCGGG GGUUC |
| | | | | | | AAAUC CCCCU CUCUC |
| | | | | | | CGCCA-3′ |

### 2-6. Preparation of M1 RNA

The first PCR (50 μL) was performed under the following conditions: 1×KOD-Plus-PCR Buffer (TOYOBO), 1.5 mM MgSO₄, 0.2 mM each of dNTPs, 0.25 μM pGEM-Tseq.R20, 0.25 μM M1RNA.R22, 8 ng of M1 RNA template plasmid, 0.05 U/μL KOD DNA polymerase (TOYOBO).

This solution was heated at 94° C. for 3 minutes and then DNA was amplified by repeating 25 cycles of 94° C. for 15 seconds, 55° C. for 30 seconds, 68° C. for 1 minute.

The second PCR (1.6 mL) was performed under the following conditions; 1×PCR buffer, 0.5 μM T7ex5.F22, 0.5 μM M1RNA.R22, 1.6 nM Phusion DNA polymerase.

This solution was heated at 94° C. for 3 minutes and then DNA was amplified by repeating 25 cycles of 94° C. for 30 seconds, 50° C. for 30 seconds, and 72° C. for 1 min.

In vitro transcription (8 mL) was performed under the following conditions: 1×T7 buffer, 30 mM MgCl₂, 20% (v/v) of the second PCR solution, 0.3 μM T7 RNA polymerase.

M1 RNA was purified by a method similar to that of purification of tRNA. The concentration of M1 RNA was measured by the absorbance at 260 nm.

2-7. Cleavage of Pre-tRNA with RNase P

M1 RNA was refolded under the following conditions: 7.5 μM M1 RNA, 1×T7 buffer, 100 mM NH₄Cl.

Solution was heated at 94° C. for 1 minute and cooled to 37° C. at 0.5° C./s and incubated it for 5 minutes. 5 mM MgCl₂ and 5 μM C5 protein were added thereto and then the solution was incubated at 25° C. for 5 minutes. 1080 μL of the obtained RNase P solution, 500 μL of 3 M NH₄Cl, 30 μL of 3 M MgCl₂, and 240 μL of DNase I (1 u/μL, Roche) were added to 12 mL of the pre-tRNA^{Gln}, pre-tRNA^{Pro}, or pre-tRNA^{Trp} solution. The solution was incubated at 37° C. for 12 hours. The tRNA was purified by the method described above. The tRNA concentration was measured by the absorbance at 260 nm.

### 2-8. Preparation of DNA Template and Expression of Peptide

The DNA template for peptide expression was prepared by the following method. Elongation (5 μL) was performed under the following conditions: 1×KOD PCR Buffer (TOYOBO), 1.5 mM MgSO4, 0.2 mM each dNTP, 1 μM T7esD6MYYY.F55, 1 μM MYYYxDDuaaAS.R38 [5′-CGAAG CTTAG TCGTC X GTAGT AGTAC ATGTT TTTCT-3′; (x, X)=(gcu, AGC), (cgu, ACG), (aau, ATT), (gau, ATC), (ugu, ACA), (cag, CTG), (gag, CTC), (ggu, ACC), (cau, ATG), (auu, AAT), (cuu, AAG), (aag, CTT), (aug, CAT), (uuu, AAA), (ccu, AGG), (ucu, AGA) (acu, AGU), (ugg, CCA), (uau, ATA), or (guu, AAC)] (see Tables below), 0.05 U/μL KOD DNA polymerase.

TABLE 6

| Primer name | Sequence |
|---|---|
| pGEM- | 5′-GGAAA CAGCT ATGAC CATGA-3′ |
| Tseq.R20 | |
| M1RNA.R22 | 5′-AGGTG AAACT GACCG ATAAG CC-3′ |
| T7pro- | 5′-GTAAT ACGAC TCACT ATAGG AACGC |
| leader.F36 | GCGAC TCTAA T-3′ |
| T7ex5.F22 | 5′-GGCGT AATAC GACTC ACTAT AG-3′ |
| T7SD8M.F48 | 5′-TAATA CGACT CACTA TAGGG TTAAC |
| | TTTAA GAAGG AGATA TACAT ATG-3′ |
| T7g10.F26 | 5′-CTAGT AATAC GACTC ACTAT AGGGT |
| | T-3′ |
| CGCStv.R20 | 5′-ACATA GTTAC TGCTG GACGG-3′ |
| NGC1Stv.R20 | 5′-ACATA GTTAC TGCTG AACGG-3′ |
| NGC2Stv.R20 | 5′-ACATA GTTAC TGCTG GACAA-3′ |

TABLE 7

| Codon | Primer name | Sequence |
|---|---|---|
| GCU | MYYYgcuDDuaaAS.R38 | 5′-CGAAG CTTAG |
| | | TCGTC AGCGT AGTAG |
| | | TACAT GTTTT TCT-3′ |
| CGU | MYYYcguDDuaaAS.R38 | 5′-CGAAG CTTAG |
| | | TCGTC ACGGT AGTAG |
| | | TACAT GTTTT TCT-3′ |
| AAU | MYYYaauDDuaaAS.R38 | 5′-CGAAG CTTAG |
| | | TCGTC ATTGT AGTAG |
| | | TACAT GTTTT TCT-3′ |
| GAU | MYYYgauDDuaaAS.R38 | 5′-CGAAG CTTAG |
| | | TCGTC ATCGT AGTAG |
| | | TACAT GTTTT TCT-3′ |
| UGU | MYYYuguDDuaaAS.R38 | 5′-CGAAG CTTAG |
| | | TCGTC ACAGT AGTAG |
| | | TACAT GTTTT TCT-3′ |
| CAG | MYYYcagDDuaaAS.R38 | 5′-CGAAG CTTAG |
| | | TCGTC CTGGT AGTAG |
| | | TACAT GTTTT TCT-3′ |
| GAG | MYYYgagDDuaaAS.R38 | 5′-CGAAG CTTAG |
| | | TCGTC CTCGT AGTAG |
| | | TACAT GTTTT TCT-3′ |
| GGU | MYYYgguDDuaaAS.R38 | 5′-CGAAG CTTAG |
| | | TCGTC ACCGT AGTAG |
| | | TACAT GTTTT TCT-3′ |
| CAU | MYYYcauDDuaaAS.R38 | 5′-CGAAG CTTAG |
| | | TCGTC ATGGT AGTAG |
| | | TACAT GTTTT TCT-3′ |
| AUU | MYYYauuDDuaaAS.R38 | 5′-CGAAG CTTAG |
| | | TCGTC AATGT AGTAG |
| | | TACAT GTTTT TCT-3′ |
| CUU | MYYYcuuDDuaaAS.R38 | 5′-CGAAG CTTAG |
| | | TCGTC AAGGT AGTAG |
| | | TACAT GTTTT TCT-3′ |
| AAG | MYYYaagDDuaaAS.R38 | 5′-CGAAG CTTAG |
| | | TCGTC CTTGT AGTAG |
| | | TACAT GTTTT TCT-3′ |
| AUG | MYYYaugDDuaaAS.R38 | 5′-CGAAG CTTAG |
| | | TCGTC CATGT AGTAG |
| | | TACAT GTTTT TCT-3′ |
| UUU | MYYYuuuDDuaaAS.R38 | 5′-CGAAG CTTAG |
| | | TCGTC AAAGT AGTAG |
| | | TACAT GTTTT TCT-3′ |
| CCU | MYYYccuDDuaaAS.R38 | 5′-CGAAG CTTAG |
| | | TCGTC AGGGT AGTAG |
| | | TACAT GTTTT TCT-3′ |
| UCU | MYYYucuDDuaaAS.R38 | 5′-CGAAG CTTAG |
| | | TCGTC AGAGT AGTAG |
| | | TACAT GTTTT TCT-3′ |
| ACU | MYYYacuDDuaaAS.R38 | 5′-CGAAG CTTAG |
| | | TCGTC AGTGT AGTAG |
| | | TACAT GTTTT TCT-3′ |
| UGG | MYYYuggDDuaaAS.R38 | 5′-CGAAG CTTAG |
| | | TCGTC CCAGT AGTAG |
| | | TACAT GTTTT TCT-3′ |
| UAU | MYYYuauDDuaaAS.R38 | 5′-CGAAG CTTAG |
| | | TCGTC ATAGT AGTAG |
| | | TACAT GTTTT TCT-3′ |
| GUU | MYYYguuDDuaaAS.R38 | 5′-CGAAG CTTAG |
| | | TCGTC AACGT AGTAG |
| | | TACAT GTTTT TCT-3′ |
| Any | T7eSD6MYYY.F55 | 5′-TAATA CGACT |
| | | CACTA TAGGG TTAAC |
| | | TTTAA CAAGG AGAAA |
| | | AACAT GTACT ACTAC-3′ |

The elongation was performed by heating the aforementioned solution at 94° C. for 3 minutes and then repeating 5 cycles of 50° C. for 1 minute and 68° C. for 1 minute.

PCR (200 μL) was performed under the following conditions: 10 mM Tris-HCl pH 8.4, 50 mM KCl, 0.1% (v/v) Triton X-100, 2 mM MgCl₂, 0.25 mM each of dNTPs, 0.5 μM T7ex5.F22, 0.5 μM each MYYYxDDuaaAS.R38, 0.05% (v/v) elongated DNA, 1% (v/v) Taq DNA polymerase.

DNA was amplified by repeating 12 cycles of 94° C. for 40 seconds, 60° C. for 40 seconds, and 72° C. for 40 seconds. After the phenol/chloroform extraction, the DNA product was collected by ethanol precipitation and dissolved in 20 μL of ultrapure water.

### 2-9. Preparation of Streptavidin Gene DNA

Streptavidin gene was synthesized by Eurofins Genomics. According to the corresponding genetic code, genes were each named CGC.stv, NGC1.stv, and NGC2.stv.

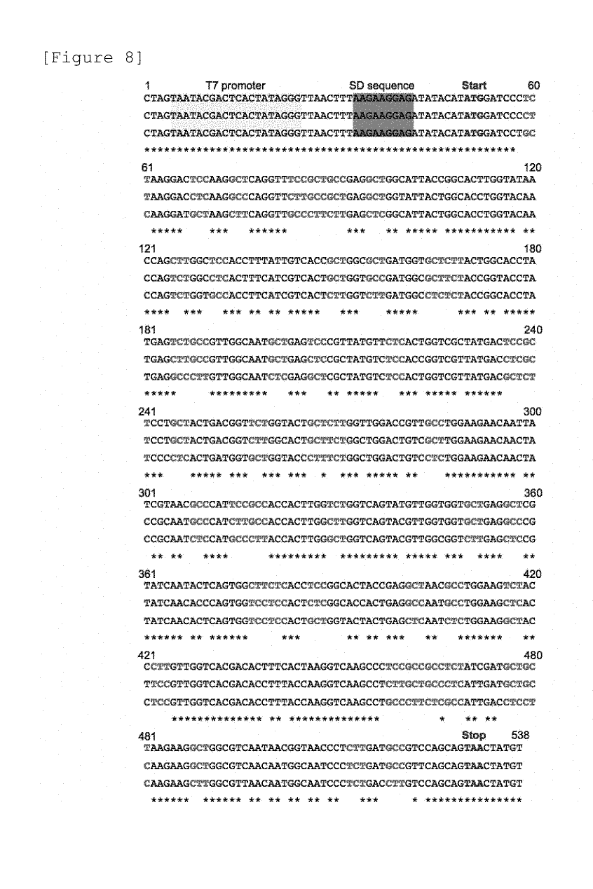

Streptavidin gene sequences are illustrated in FIG. 8.

The first PCR (5 μL) was performed under the following conditions: 1×KOD-Plus-PCR Buffer, 1.5 mM MgSO₄, 0.2 mM each of dNTPs, 2 ng of CGC.stv, NGC1.stv, or NGC2.stv template plasmid, 1 μM T7g10M.F48, 1 μM CGCStv.R20, NGC1Stv.R20, or NGC2Stv.R20, 0.05 U/μL of KOD DNA polymerase.

This solution was heated at 94° C. for 3 minutes and then DNA was amplified by repeating 15 cycles of 94° C. for 20 seconds, 55° C. for 30 seconds, and 68° C. for 1 min.

The second PCR (10 μL) was performed under the following conditions; 1×KOD-Plus-PCR Buffer, 1.5 mM MgSO4, 0.2 mM each of dNTPs, 0.025% (v/v) of the first PCR solution, 1 μM T7g10.F26, 1 μM CGCStv.R20, NGC1Stv.R20, or NGC2Stv.R20, 0.05 U/μL of KOD DNA polymerase.

This solution was heated at 94° C. for 3 minutes and then DNA was amplified by repeating 15 cycles of 94° C. for 20 seconds, 55° C. for 30 seconds, and 68° C. for 1 min.

The third PCR (8 mL) was performed under the following conditions: 1×PCR buffer, 0.05% (v/v) of the second PCR solution, 1 μM T7g10.F26, 1 μM CGCStv.R20, NGC1Stv.R20, or NGC2Stv.R20, 1.6 nM Phusion DNA polymerase.

This solution was heated at 94° C. for 3 minutes and then DNA was amplified by repeating 12 cycles of 94° C. for 20 seconds, 55° C. for 30 seconds, and 72° C. for 1 min. After the phenol/chloroform extraction, the DNA product was collected by 2-propanol precipitation and dissolved in 200 μL of ultrapure water.

2-10. Analysis of Activity and Accuracy of Native and in Vitro Transcribed tRNA

The reconstituted cell-free translation system that does not contain 20 proteinous amino acids was prepared according to a known method. In vitro transcribed tRNA^{fMet}, tRNA^{Asp}, tRNA^{Tyr}, and tRNA^{Xaa} were mixed and diluted 5 times. After heating at 95° C. for 3 minutes, the tRNAs were precipitated by ethanol precipitation and dissolved in ultrapure water.

Translation (1 μL) was performed under the following conditions: 20% (v/v) of DNA template solution, 250 μM each of 19 proteinous amino acids (other than Asp), 50 μM [¹⁴C]-Asp and 1.5 μ/μL native tRNA or 12 μM each of in vitro transcribed tRNA^{fMet}, tRNA^{Asp}, tRNA^{Tyr}, and tRNA^{Xaa}.

In the analysis of the accuracy of translation, the amino acid to which the x codon is assigned was removed from the DNA template in each reaction solution. The concentrations of other components are set forth in Table 2 above.

The solution was incubated at 37° C. for 2 hours. The obtained product was analyzed by Tricine-SDS-PAGE and autoradiography (FLA-5100, Fuji). Moreover, a similar experiment was conducted by using Asp instead of [¹⁴C]-Asp and the obtained product was measured according to a known method with autoflex II (BRUKER DALTONICS).

### 2-11. Orthogonal Expression of Streptavidin

For the universal genetic code, new genetic code 1, and new genetic code 2, 21 in vitro transcribed tRNAs were prepared. In vitro transcription (1.5 μL) was performed under the following conditions: 20% (v/v) streptavidin gene, 250 μM each of 19 proteinous amino acids (other than Asp), 50 μM [¹⁴C]-Asp, Asp, and 1.5 μg/μL native tRNA or 12 μM each of in vitro transcribed tRNAs.

The solution was incubated at 37° C. for 2 hours. The obtained product was analyzed by Tricine-SDS-PAGE and autoradiography. Moreover, a similar experiment was conducted by using Asp instead of [¹⁴C]-Asp. Asp. The obtained product was diluted 20 times with TBST (25 mM Tris-HCl pH 7.5, 150 mM NaCl, 0.1% Tween 20) containing 0.01% (w/v) BSA. 2 μL of the product after the dilution was spotted (triplicate) on a nitrocellulose membrane (GE Healthcare UK Ltd.). The membrane was blocked with TBST containing 0.2% (w/v) BSA for 10 minutes and then labeling with 15 μg of biotin conjugated horseradish peroxidase (Invitrogen, ultrafiltered three times) in TBST containing 0.2% (w/v) BSA was conducted for 30 minutes. The membrane was washed three times with TBST for 5 minutes. To detect the streptavidin bound to the biotin, the nitrocellulose membrane was incubated with Luminata™ Western HRP Substrate (MILLIPORE). The fluorescence was measured using Ez-Capture MG (ATTO). The fluorescence intensity of each spot was analyzed using Image J.

### 3. Result

3-1. Preparation of In Vitro Transcribed tRNAs and Evaluation of their Activity in Translation

tRNAs (Table 5) corresponding to 20 proteinous amino acids were prepared by in vitro transcription. Moreover, 3 tRNAs (tRNA^{Trp}, tRNA^{Gln}, tRNA^{Pro}) were prepared with an additional RNase P13 treatment since bases at position 1 and 72 in these tRNAs are involved in the recognition by the aminoacyl-tRNA synthetases. As a general rule, the in vitro transcribed tRNAs were designed based on the sequences of the native tRNAs in the Escherichia coli strain K12 and some of the tRNAs were mutated as described below. Since the thiouridine derivative of U34 in tRNA^{Lys}_{UUU} and tRNA^{Glu}_{UUC} is important for translation, the wobble position 34 in tRNA^{Lys}_{UUU} and tRNA^{Glu}_{UUC} was changed from U to C. This is known to markedly reduce the k_{cat}/K_{M} of aminoacylation but restore the translation activity of the right codon. Moreover, by mutating 1U72A of tRNA^{Asn}_{GUU} and 1A72U of tRNA^{Ile}_{GAU} into 1G72C and 1C of tRNA^{fMet}_{CAU} into 1G, these tRNAs were prepared by in vitro transcription without engineering RNase P. Furthermore, tRNA^{Arg}_{GCG} was prepared by mutating A34 of tRNA^{Arg}_{ACG} into G34. To construct translation systems using codon-shuffled genetic codes 1 and 2 set forth in FIGS. 2B and 2C, chimeric tRNA^{Ala}, tRNA^{Leu}, and tRNA^{Ser} in which the whole anticodon loops, but not only the anticodons, have been exchanged were prepared. This design is based on the knowledge that the nucleotide sequence of the anticodon loop is important in accuracy of translation by tRNA.

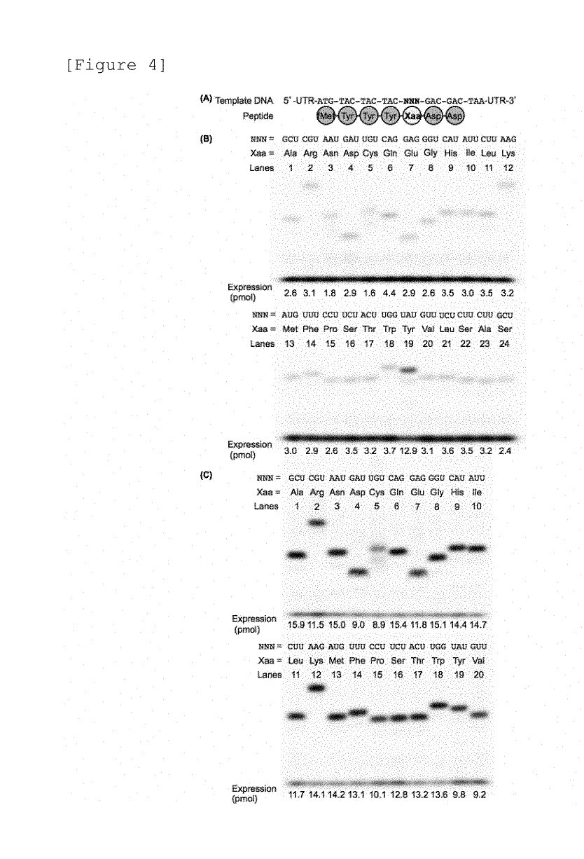

To examine the ability of these in vitro transcribed tRNAs to decode right codons in the reconstituted translation system, the following peptide expression assay was performed: in vitro transcribed tRNA^{fMet}_{CAU}, tRNA^{Asp}_{GUC}, tRNA^{Tyr}_{GUA}, tRNA^{Xaa} and a peptide template 5′-UTR-ATG-(TAC)₃-NNN-(GAC)₂-TAA-UTR-3′ (UTR denotes a untranslated region and Xaa denotes the amino acid corresponding to the NNN codon) (FIG. 4A). If the right NNN codon is decoded by tRNAXaa in the presence of [¹⁴C]-Asp, then the [¹⁴C]-Asp labeled peptide Met-(Tyr)₃-Xaa-(Asp)₂ are produced. In fact, it was confirmed by the analysis using Tricine-SDS-PAGE and autoradiography that the peptide was expressed from each template DNA (FIG. 4B).

Except the case where a template in which the position of NNN is the TAC codon is used, a single band was found in the gel. In the case of the TAC codon, in addition to an expected main band, a small band, which is considered to be generated by an error of tRNA^{Tyr}_{GUA} in decoding the UAA stop codon, was found. More importantly, the mobility of each peptide was the same as the peptide translated with native tRNAs (FIG. 4C). To confirm that the corresponding amino acids are present in the peptides, the template DNA was translated in the same way using Asp instead of [¹⁴C]-Asp and the translated products were analyzed by MALDI-TOF-MS. As expected, the mass of the expressed peptides was same as the calculated mass. The mobility in Tricine-SDS-PAGE and the mass spectrometry data from MALDI-TOF-MS indicated that the in vitro transcribed tRNAs can translate right codons.

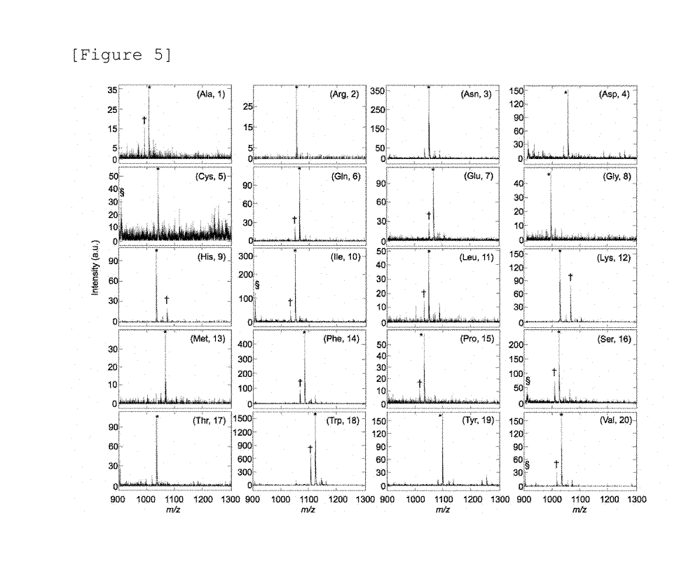

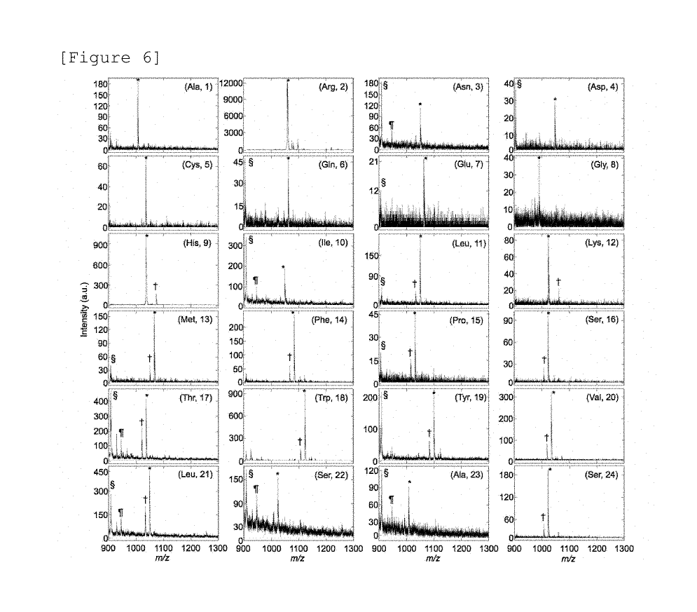

To examine ability of chimeric tRNAs to translate, similar experiments were conducted using chimeric tRNA^{Leu}_{GGA} tRNA^{Ser}_{GAG}, tRNA^{Ala}_{GAG}, tRNA^{Ser}_{GCC} and the corresponding DNA templates. From the result of Tricine-SDS-PAGE and the mass spectrometric result in MALDI-TOF-MS, it was indicated that the expected peptides were expressed in all translation reactions (FIGS. 4 to 6). In this way, it was revealed that use of tRNA^{Leu}_{GGA}, tRNA^{Ser}_{GAG}, tRNA^{Ala}_{GAG}, and tRNA^{Ser}_{GCC} results in the assignment of the UCC codon to Leu, the CUC codon to Ser, the CUC codon to Ala, and the GGC codon to Ser.

3-2. Accuracy of Translation with the Native tRNA Set and with In Vitro Transcribed tRNA Sets

Since the bases of the in vitro transcribed tRNAs are not modified, decrease in accuracy of translation with the in vitro transcribed tRNAs in comparison with the native tRNAs was feared. To confirm the possibility, the error of the native tRNA set and in vitro transcribed tRNA sets in decoding was examined.

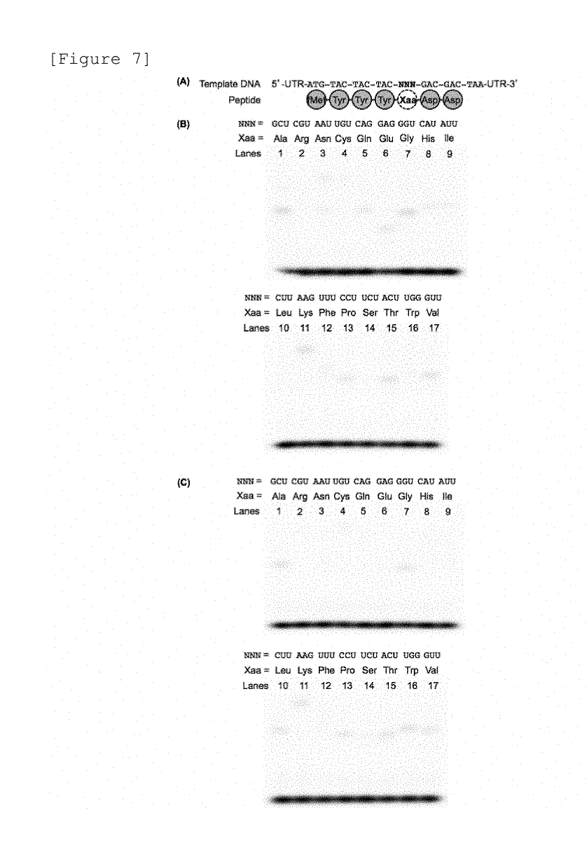

First, for the translation using the universal genetic code, 20 in vitro transcribed tRNAs corresponding to the 20 proteinous amino acids and tRNA^{fMet} were used in mixture as an in vitro transcribed tRNA set. As the native tRNA set, tRNA extracted from E. coli MRE600 was used. These RNA sets were added to a reconstituted translation system without tRNA and peptide translation was performed in the absence of Xaa corresponding to the NNN codon (FIG. 7). In this translation reaction, if the tRNA set translates only right codons accurately, then no peptides should be expressed, while if the accuracy of the translation is low and wrong codons are translated, then some peptides should be expressed.

In the result, expression of some peptides was found with the native tRNAs. This indicates that the native tRNAs make translation errors from some codons in this extreme condition. From the mobility of these bands and the result of mass spectrometry, it was found that Ser was incorporated instead of Ala by mistake, Met was incorporated instead of Pro by mistake, and Asn was incorporated instead of Val by mistake (data not shown). These results suggested that tRNA^{Ser}_{GGA} makes error in translating GCU, tRNA^{Met}_{CAU} makes error in translating CCU, and tRNA^{Asn}_{GUU} makes error in translating GUU.

Therefore, the accuracy of translation with in vitro transcribed tRNA sets was then examined using the same assay system. The result of Tricine-SDS-PAGE analysis indicated that translation error of some codons occurs at the same rate as that with the native tRNA set (FIG. 7).

From the foregoing results, it was concluded that the in vitro transcribed tRNA sets have a similar accuracy to that of the native tRNAs and can be used in protein expression.

### 3-3. Expression of Streptavidin by Decoding Universal Genetic Code

Streptavidin was chosen as a model protein and a gene encoding streptavidin according to the universal genetic code was constructed.

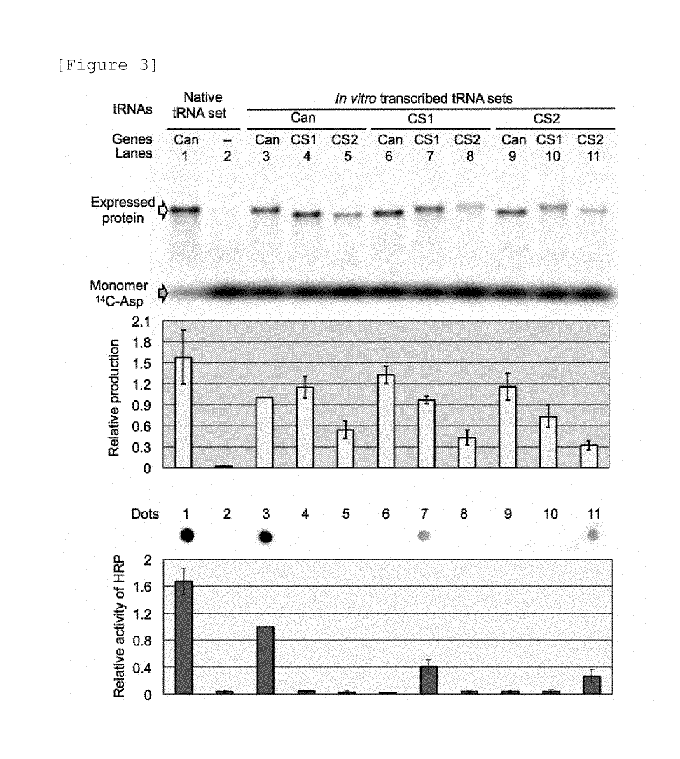

As control, translation using the native tRNAs and [¹⁴C]-Asp Asp was performed in the presence or absence of the universal gene. By Tricine-SDS-PAGE analysis, it was revealed that the streptavidin is expressed in the presence of the universal gene (FIG. 3, Lane 1) and no protein is expressed in the absence of the universal gene (FIG. 3, Lane 2).

The biotin binding activity of the streptavidin was examined by also conducting the dot blot assay using biotin conjugated horseradish peroxidase (biotin-HRP). In the assay, the binding activity was found in the presence of the gene (FIG. 3, dot 1), but not in the absence of the gene (FIG. 3, dot 2). From these results, it was indicated that active streptavidin is produced from the universal streptavidin gene in this translation system.

Next, the universal streptavidin gene was translated using the universal in vitro transcribed tRNA set (the universal tRNA set, tRNA No. 0 to 20 in Table 5). In comparison with the case with the native tRNA set, the production rate of the streptavidin is decreased by approximately 70% (FIG. 3, Lane 3), which was considered to be caused by the lack of the base modification in the in vitro transcribed tRNA set. Importantly, the biotin binding activity was found in the presence of the gene (FIG. 3, dot 3), which suggested that accurate translation was conducted.

### 3-4. Expression of Streptavidin by Decoding Codon-Shuffled Genetic Code

To decode codon-shuffled genetic code 1 (CS1 tRNA set), an in vitro transcribed tRNA set using tRNA^{Leu}_{GGA} (tRNA No. 21) and tRNA^{Ser}_{GAG} (tRNA No. 22) instead of tRNA^{Leu}_{GAG} (tRNA No. 11 in Supplementary Table 1) and tRNA^{Ser}_{GGA} (tRNA No. 16) was then prepared.

Since the UCU and UCC codons were assigned to Leu and the CUU and CUC codons were assigned to Ser in the translation system using the CS1 tRNA set (FIG. 2B), this means that all the Leu and Ser codons in the streptavidin gene were interchanged (see CS1 gene in FIG. 8).

By the translation of the CS1 gene using the CS1 tRNA set in the translation system, the streptavidin was produced in an amount at the same level as the amount in the case with the universal gene and the universal tRNA set (FIG. 3, Lane 7). The biotin binding activity was also observed, but the intensity was slightly decreased (FIG. 3, Lane 7).

Furthermore, an in vitro transcribed tRNA set (CS2 tRNA set) for codon-shuffled genetic code 2 was prepared using tRNA^{Ala}_{GAG} (tRNA No. 23), tRNA^{Leu}_{GGA} (tRNA No. 21), and tRNA^{Ser}_{GGC} (tRNA No. 24) instead of tRNA^{Ala}_{GGC} (tRNA No. 1), tRNA^{Leu}_{GAG} (tRNA No. 11) and tRNA^{Ser}_{GGA} (tRNA No. 16).

In this translation system using the CS2 tRNA set, the CUU and CUC codons were assigned to Ala, the UCU and UCC codons were assigned to Leu, and the GCU and GCC codons were assigned to Ser (FIG. 2C). Therefore, all the Leu, Ser, and Ala codons in the streptavidin gene were interchanged (see CS2 gene in FIG. 8).

By the translation of the CS2 gene in the translation system using the CS2 tRNA set, the streptavidin was produced in an amount equal to approximately one-third of the amount in the case with the universal gene and the universal tRNA set (FIG. 3, Lane 11, dot 11). From these experimental results, it was indicated that active streptavidin is expressed with the codon-shuffled genetic code.

### 3-5. Orthogonal Expression of Streptavidin by Decoding Codon-Shuffled Genetic Code

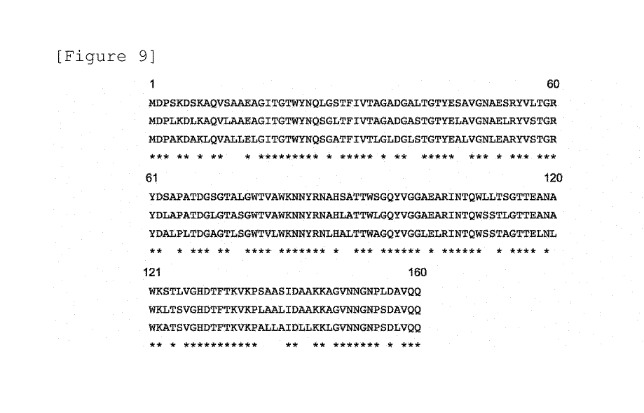

One of the most important aspects of the present invention is orthogonality of genes encoded by codon-shuffled genetic codes. To demonstrate orthogonality of codon-shuffled genetic code 1 to the universal genetic code, a CS1 gene was translated in a translation system using the universal tRNA set. By the Tricine-SDS-PAGE analysis, it was indicated that the protein was expressed from the CS1 gene (FIG. 3, Lane 4). Since there are 14 codons that specify Ser and 8 codons that specify Leu in the streptavidin gene, 22 amino acid substitutions should occur in the produced protein (FIG. 9), which was considered to be sufficient mutations to reduce the biotin binding activity of the protein. As expected, no biotin binding activity was found in the product obtained by translating the CS1 gene in the translation system using the universal tRNA set (FIG. 3, dot 4).

Furthermore, the CS2 gene was similarly translated using the universal tRNA set to examine the orthogonality of codon-shuffled genetic code 2 to the universal genetic code. Since the streptavidin gene has 25 codons that specify Ala, the produced protein should have 47 amino acid substitutions in total (FIG. 9). In fact, the protein was expressed, but the biotin binding activity was not found (FIG. 3, Lane 5, dot 5).

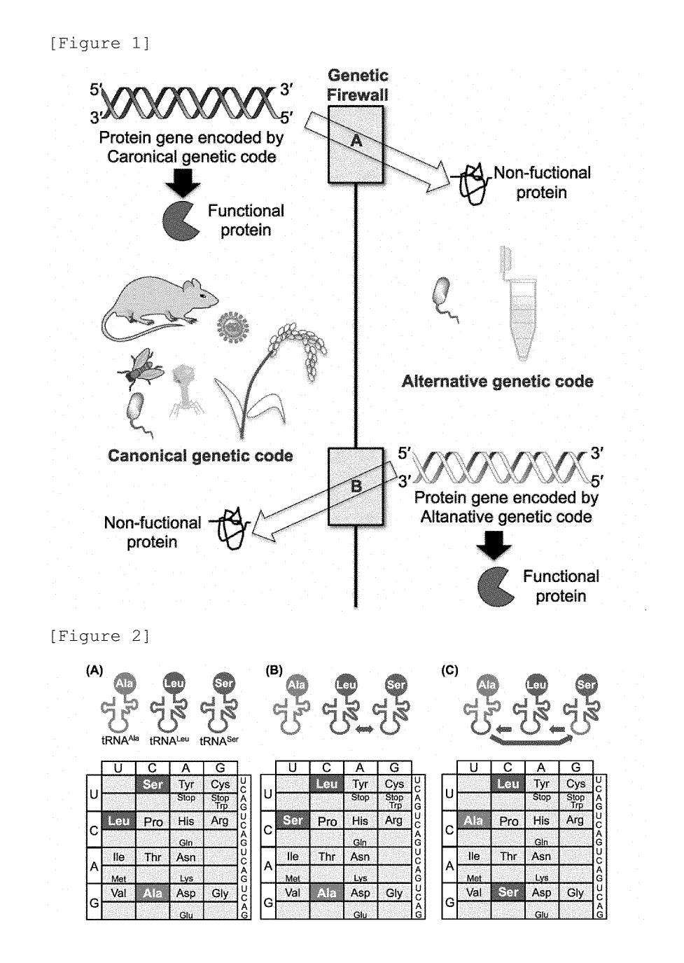

From the foregoing results, it was demonstrated that the orthogonal expression of CS1 and CS2 genes was obtained, that is, genetic firewall was formed (FIG. 1).

1. A translation system for expressing a polypeptide having a predetermined amino acid sequence according to a code different from the universal genetic code, comprising
a template nucleic acid having a sequence modified from a nucleic acid sequence encoding the polypeptide by interchanging codons encoding at least 2 amino acids selected from serine, leucine, and alanine; and
at least either (i) or (ii):
(i) an aminoacyl-tRNA having an anticodon to a codon after interchange and to which an amino acid encoded by a codon before the interchange is bound;
(ii) a tRNA having an anticodon to a codon after interchange and recognized by an aminoacyl-tRNA synthetase corresponding to an amino acid encoded by a codon before the interchange, or a nucleic acid encoding the tRNA, the amino acid, and the aminoacyl-tRNA synthetase or a nucleic acid encoding the aminoacyl-tRNA synthetase.

2. The translation system according to claim 1, wherein the template nucleic acid has a sequence modified from the nucleic acid sequence encoding the polypeptide by interchanging a codon encoding serine and a codon encoding leucine, and the translation system comprises
at least either (i) or (ii) and
at least either (iii) or (iv):
(i) an aminoacyl-tRNA having an anticodon to a codon encoding leucine in the universal genetic code and to which serine is bound;
(ii) a tRNA having an aminoacyl-anticodon to a codon encoding leucine in the universal genetic code and recognized by a seryl-tRNA synthetase or a nucleic acid encoding the tRNA, serine, and the seryl-tRNA synthetase or a nucleic acid encoding the seryl-tRNA synthetase;
(iii) an aminoacyl-tRNA having an anticodon to a codon encoding serine in the universal genetic code and to which leucine is bound;
(iv) a tRNA having an anticodon to a codon encoding serine in the universal genetic code and recognized by a leucyl-tRNA synthetase or a nucleic acid encoding the tRNA, leucine, and the leucyl-tRNA synthetase or a nucleic acid encoding the leucyl-tRNA synthetase.

3. The translation system according to claim 1, wherein the template nucleic acid has a sequence modified from the nucleic acid sequence encoding the polypeptide by interchanging a codon encoding serine and a codon encoding alanine, and the translation system comprises
at least either (i) or (ii) and
at least either (iii) or (iv):
(i) an aminoacyl-tRNA having an anticodon to a codon encoding alanine in the universal genetic code and to which serine is bound;
(ii) a tRNA having an anticodon to a codon encoding alanine in the universal genetic code and recognized by a seryl-tRNA synthetase or a nucleic acid encoding the tRNA, serine, and the seryl-tRNA synthetase or a nucleic acid encoding the seryl-tRNA synthetase;
(iii) an aminoacyl-tRNA having an anticodon to a codon encoding serine in the universal genetic code and to which alanine is bound;
(iv) a tRNA having an anticodon to a codon encoding serine in the universal genetic code and recognized by an alanyl-tRNA synthetase or a nucleic acid encoding the tRNA, alanine, and the alanyl-tRNA synthetase or a nucleic acid encoding the alanyl-tRNA synthetase.

4. The translation system according to claim 1, wherein the template nucleic acid has a sequence modified from the nucleic acid sequence encoding the polypeptide by interchanging a codon encoding leucine and a codon encoding alanine, and the translation system comprises
at least either (i) or (ii) and
at least either (iii) or (iv):
(i) an aminoacyl-tRNA having an anticodon to a codon encoding alanine in the universal genetic code and to which leucine is bound;
(ii) a tRNA having an anticodon to a codon encoding alanine in the universal genetic code and recognized by a leucyl-tRNA synthetase or a nucleic acid encoding the tRNA, leucine, and the leucyl-tRNA synthetase or a nucleic acid encoding the leucyl-tRNA synthetase;
(iii) an aminoacyl-tRNA having an anticodon to a codon encoding leucine in the universal genetic code and to which alanine is bound;
(iv) a tRNA having an anticodon to a codon encoding leucine in the universal genetic code and recognized by an alanyl-tRNA synthetase or a nucleic acid encoding the tRNA, alanine, and the alanyl-tRNA synthetase or a nucleic acid encoding the alanyl-tRNA synthetase.

5. The translation system according to claim 1, wherein the template nucleic acid has a sequence modified from the nucleic acid sequence encoding the polypeptide by exchanging a codon encoding serine for a codon encoding leucine, a codon encoding leucine for a codon encoding alanine, and a codon encoding alanine for a codon encoding serine, and the translation system comprises;
at least either (i) or (ii),
at least either (iii) or (iv), and
at least either (v) or (vi):
(i) an aminoacyl-tRNA having an anticodon to a codon encoding leucine in the universal genetic code and to which serine is bound;
(ii) a tRNA having an anticodon to a codon encoding leucine in the universal genetic code and recognized by a seryl-tRNA synthetase or a nucleic acid encoding the tRNA, serine, and the seryl-tRNA synthetase or a nucleic acid encoding the seryl-tRNA synthetase;
(iii) an aminoacyl-tRNA having an anticodon to a codon encoding alanine in the universal genetic code and to which leucine bound;
(iv) a tRNA having an anticodon to a codon encoding alanine in the universal genetic code and recognized by a leucyl-tRNA synthetase or a nucleic acid encoding the tRNA, leucine, and the leucyl-tRNA synthetase or a nucleic acid encoding the leucyl-tRNA synthetase;
(v) an aminoacyl-tRNA having an anticodon to a codon encoding serine in the universal genetic code and to which alanine bound;
(vi) a tRNA having an anticodon to a codon encoding serine in the universal genetic code and recognized by an alanyl-tRNA synthetase or a nucleic acid encoding the tRNA, alanine, and the alanyl-tRNA synthetase or a nucleic acid encoding the alanyl-tRNA synthetase.

6. The translation system according to claim 1, wherein the template nucleic acid has a sequence modified from the nucleic acid sequence encoding the polypeptide by exchanging a codon encoding serine for a codon encoding alanine, a codon encoding alanine for a codon encoding leucine, and a codon encoding leucine for a codon encoding serine, and the translation system comprises;
at least either (i) or (ii),
at least either (iii) or (iv), and
at least either (v) or (vi):
(i) an aminoacyl-tRNA having an anticodon to a codon encoding alanine and to which serine bound;
(ii) a tRNA having an anticodon to a codon encoding alanine in the universal genetic code and recognized by a seryl-tRNA synthetase or a nucleic acid encoding the tRNA, serine, and the seryl-tRNA synthetase or a nucleic acid encoding the seryl-tRNA synthetase;
(iii) an aminoacyl-tRNA having an anticodon to a codon encoding leucine in the universal genetic code and to which alanine bound;
(iv) a tRNA having an anticodon to a codon encoding leucine in the universal genetic code and recognized by an alanyl-tRNA synthetase or a nucleic acid encoding the tRNA, alanine, and the alanyl-tRNA synthetase or a nucleic acid encoding the alanyl-tRNA synthetase.
(v) an aminoacyl-tRNA having an anticodon to a codon encoding serine in the universal genetic code and to which leucine bound;
(vi) a tRNA having an anticodon to a codon encoding serine in the universal genetic code and recognized by a leucyl-tRNA synthetase or a nucleic acid encoding the tRNA, leucine, and the leucyl-tRNA synthetase or a nucleic acid encoding the leucyl-tRNA synthetase.

7. A cell comprising the translation system according to claim 1.

8. A living organism comprising the translation system according to claim 1.

9. A method for producing a polypeptide having a predetermined amino acid sequence, comprising a step of:
expressing the polypeptide from the template nucleic acid in the translation system according to claim 1.

10. A kit comprising
the translation system according to claim 1, whereby the template nucleic acid is packaged together with (i) the aminoacyl-tRNA, (ii) the tRNA having the anticodon, or both.
